(19) United States
(12) Patent Application Publication
(10) Pub. No.: US 2018/0046774 A1
(43) Pub. Date: Feb. 15, 2018

(54) METHOD FOR DETERMINING GASTROINTESTINAL TRACT DYSBIOSIS

(71) Applicant: GENETIC ANALYSIS AS, OSLO (NO)

(72) Inventors: TORBJORN LINDAHL, OSLO (NO); MAGDALENA KARLSSON, OSLO (NO); MONIKA SEKELJA, OSLO (NO); FINN HEGGE, OSLO (NO)

(21) Appl. No.: 15/560,564

(22) PCT Filed: Mar. 24, 2016
(86) PCT No.: PCT/EP2016/056670

§ 371 Date: Sep. 22, 2017

(30) Foreign Application Priority Data
Mar. 27, 2015 (GB) 1505364.8

(51) Int. Cl. G06F 19/00; A61B 5/00

(52) U.S. Cl. CPC G06F 19/3431; G06F 19/345; A61B 5/42; A61B 5/7275

(57) ABSTRACT

The invention provides a method for determining the likelihood of GI tract dysbiosis in a subject, said method comprising providing a test data set, wherein said test data set comprises at least one microbiota profile, said microbiota profile being a profile of the relative levels of a plurality of microorganisms or groups of microorganisms in a sample from the GI tract of the subject and wherein each level of each microorganism or group of microorganisms is a profile element of said test data set, applying to said test data set at least one loading vector determined from latent variables within the profiles of the levels of said plurality of microorganisms or groups of microorganisms in corresponding GI tract samples from a plurality of normal subjects, thereby producing a first projected data set, applying to said first projected data set a transposed version of said at least one loading vector, thereby producing a second projected data set, comparing said test data set with said second projected data set and combining the differences between the corresponding profile elements of the second projected data set and the test data set and comparing the combined differences with a normobiotic to dysbiotic threshold value determined from the corresponding analysis of said plurality of microorganisms or groups of microorganisms in corresponding GI tract samples from a plurality of normal subjects and/or subjects with dysbiosis, applying at least one eigenvalue to said first projected data set, said eigenvalue determined from said at least one loading vector, and combining the resulting values for each profile element and comparing the combined values with a normobiotic to dysbiotic threshold value determined from the corresponding analysis of said plurality of microorganisms or groups of microorganisms in corresponding GI tract samples from a plurality of normal subjects and/or subjects with dysbiosis, wherein a microbiota profile with said combined differences or said combined resulting values in excess of said respective normobiotic to dysbiotic thresholds is indicative of a likelihood of dysbiosis.

The present invention concerns the diagnosis, monitoring and/or characterisation of diseases and conditions associated with perturbations in the microbiota of the gastrointestinal (GI) tract. More specifically the invention provides means by which the state of the microbiota of the GI tract may be assessed and deviations from the normal state (normobiosis), i.e. dysbiosis, may be determined in a manner which is straightforward to perform, reliable and robust and which is flexible enough to be used with any technique for measuring levels of microorganisms in a GI tract sample. In more specific embodiments such deviations may be to an extent quantified and thus the invention provides the means of determining the extent of GI tract dysbiosis, which may in turn indicate the severity of the associated disease or condition or may be used to monitor the progression of or characterise the associated disease or condition.

The GI tract, also referred to as the digestive tract or alimentary canal (and which terms may be used interchangeably with GI tract), is the continuous series of organs beginning at the mouth and ending at the anus. Throughout its length the GI tract is colonised by microorganisms of a variety of different species. Together the microorganism content of the GI tract is the microbiota of the GI tract. The relative amounts of the constituent microorganisms or groups thereof can be considered to be a profile of the microbiota. Microbiota profiles therefore give information on microbial diversity (i.e. the number of taxonomically distinct microbes or distinct taxonomic groups which are present) in the GI tract as well as providing information on the relative amounts of the microbes or groups thereof which are present.

Many diseases and conditions, or stages thereof, are believed to be associated with perturbations in the microbiota of the GI tract, or regions thereof. In some instances the disease or condition may be caused by, or is exacerbated by, the shift in the profile of the microbiota of the GI tract, or regions thereof (i.e. the relative amounts of constituent microbes and the diversity of those microbes). In other instances the disease or condition causes, or by some mechanism results in, the display of a profile of the microbiota of the GI tract that differs from the normal state. In some contexts this may even be an adaptive response attempting to address the pathological phenotype of the disease or condition. Accordingly, by assessing the state of the microbiota of the GI tract and determining deviations from the normal state (normobiosis), i.e. dysbiosis, information can be provided that permits the diagnosis, monitoring and/or characterisation of diseases and conditions associated with perturbations in the microbiota of the gastrointestinal (GI) tract or that permits, or at least is useful in, an assessment of the risk of developing a disease or condition which has been determined to be associated by a perturbation of the microbiota profile.

Diseases and conditions affecting the GI tract are very likely to result in microbiota profiles that vary from the normal state, e.g. Inflammatory Bowel Disease (IBD), Crohn's Disease (CD), Ulcerative Colitis (UC), Irritable Bowel Syndrome (IBS), small bowel bacterial overgrowth syndrome and GI tract cancers (e.g. cancer of the mouth, pharynx, oesophagus, stomach, duodenum, jejunum, ileum, cecum, colon, rectum or anus) and evidence also exists of links between GI tract microbiota profiles and diseases and conditions that are considered to be unrelated to the GI tract, for instance breast cancer; ankylosing spondylitis; non-alcoholic steatohepatitis; the atopic diseases, e.g. eczema, asthma, atopic dermatitis, allergic conjunctivitis, allergic rhinitis and food allergies; metabolic disorders, e.g. diabetes mellitus (type 1 and type 2), obesity and metabolic syndrome; neurological disorders, e.g. depression, multiple sclerosis, dementia, and Alzheimer's disease; autoimmune disease (e.g. arthritis); malnutrition; chronic fatigue syndrome and autism. It is believed that such perturbations of the GI tract microbiota profile (in terms of relative amounts and/or diversity), which may be considered to equate to an imbalance in the GI tract microbiota, contribute to these diseases, either by causing the diseases or contributing to their progression. It is also believed that many more diseases will be found to have causal links to perturbations of the GI tract microbiota profile. The precise mechanism behind this causation is not well understood. It is clear that perturbation of the microbiota of the GI tract results in the underpopulation of certain microbes and/or the overpopulation of others and/or reductions in diversity and this causes a shift, or imbalance, in the relative activities of each microbe population. It is believed that this shift in microbial activities causes a reduction in beneficial effects (e.g. synthesis of vitamins, short-chain fatty acids and polyamines, nutrient absorption, inhibition of pathogens, metabolism of plant compounds) to occur and/or an increase in deleterious effects (secretion of endotoxins and other toxic products) to occur with consequent overall negative effects on the host's overall physiology. These effects can then manifest as illness and disease, e.g. those recited above.

Although it is now common place to determine and even quantify the relative amounts of microorganisms in a GI tract sample and to use such profiles to diagnose disease by reference to specific profiles characteristic of a disease state or to rule out a diagnosis by reference to specific profiles characteristic of a normal state (e.g. WO2012080754; WO2011043654) there remains a need for methods which determine the likelihood that a patient has GI tract dysbiosis, vis a vis a normobiotic state, which are straightforward to perform, reliable and robust and which are flexible enough to be used with any technique for measuring levels of microorganisms in a GI tract sample and which do not require reference to specific standard profiles.

Thus in a first aspect the invention provides a method for determining the likelihood of GI tract dysbiosis in a subject, said method comprising:
(i) providing a test data set, wherein said test data set comprises at least one microbiota profile, said microbiota profile being a profile of the relative levels of a plurality of microorganisms or groups of microorganisms in a sample from the GI tract of the subject and wherein each level of each microorganism or group of microorganisms is a profile element of said test data set,
(ii) applying to said test data set at least one loading vector determined from latent variables within the profiles of the levels of said plurality of microorganisms or groups of microorganisms in corresponding GI tract samples from a plurality of normal subjects, thereby producing a first projected data set,
(iii) applying to said first projected data set a transposed version of said at least one loading vector, thereby producing a second projected data set,
(iv) comparing said test data set with said second projected data set and combining the differences between the corresponding profile elements of the second projected data set and the test data set and comparing the combined differences with a normobiotic to dysbiotic threshold value determined from the corresponding analysis of said plurality of microorganisms or groups of microorganisms in corresponding GI tract samples from a plurality of normal subjects and/or a plurality of subjects with dysbiosis,
(v) applying at least one eigenvalue to said first projected data set, said eigenvalue determined from said at least one loading vector, and combining the resulting values for each profile element and comparing the combined values with a normobiotic to dysbiotic threshold value determined from the corresponding analysis of said plurality of microorganisms or groups of microorganisms in corresponding GI tract samples from a plurality of normal subjects and/or subjects with dysbiosis,
   wherein step (v) may be performed before or after or concurrently with either of steps (iii) or (iv), and wherein a microbiota profile with said combined differences or said combined resulting values in excess of said respective normobiotic to dysbiotic thresholds is indicative of a likelihood of dysbiosis.

In other embodiments a likelihood of dysbiosis is indicated if both said combined differences and said combined resulting values are in excess of their respective normobiotic to dysbiotic thresholds.

The method of the invention may also be considered to be a method to identify dysbiosis, to detect dysbiosis, to determine the presence of dysbiosis or characterise dysbiosis. The method of the invention may therefore be considered to comprise a step of determining the likelihood of GI tract dysbiosis, identifying dysbiosis, detecting dysbiosis, determining the presence of dysbiosis or characterising dysbiosis in the subject based on the indication of a likelihood of dysbiosis provided in the preceding steps. The results of such a step may be recorded and optionally stored on a suitable recording/storage medium and/or communicated to a physician, the subject or intermediary or agent thereof.

In certain embodiments the method is performed on a plurality of microbiota profiles. These profiles may be from the same subject, e.g. as parallel profiles obtained from the same sample, from different corresponding samples from the same subject, e.g. obtained from the subject at different times, or from different samples from the said subject. Alternatively, or in addition, corresponding samples from another subject may be analysed together with samples from the first subject. In these embodiments it may be convenient to arrange said microbiota profiles in a test data matrix, wherein each distinct profile element is aligned across the plurality of microbiota profiles.

In a particular example of these embodiments there is provided a method for determining the likelihood of GI tract dysbiosis in a subject, said method comprising:
(i) providing a plurality of microbiota profiles, wherein each of said microbiota profiles is a profile of the relative levels of a plurality of microorganisms or groups of microorganisms in
   (a) a sample from the GI tract of the subject,
   (b) different corresponding samples from the GI tract of said subject, wherein each microbiota profile has been prepared in essentially the same way and wherein each level of each microorganism or group of microorganisms is a profile element, and arranging said microbiota profiles in a test data matrix, wherein each distinct profile element is aligned across the plurality of microbiota profiles,
(ii) applying to said test data matrix at least one loading vector determined from latent variables within the profiles of the levels of said plurality of microorganisms or groups of microorganisms in corresponding GI tract samples from a plurality of normal subjects, thereby producing a first projected data matrix,
(iii) applying to said first projected data matrix a transposed version of said at least one loading vector, thereby producing a second projected data matrix,
(iv) comparing said test data matrix with said second projected data matrix and, for each microbiota profile, combining the differences between the corresponding profile elements of the second projected data matrix and the test data matrix and comparing the combined differences in each microbiota profile with a normobiotic to dysbiotic threshold value determined from the corresponding analysis of said plurality of microorganisms or groups of microorganisms in a corresponding GI tract sample from a plurality of normal subjects and/or subjects with dysbiosis,
(v) applying at least one eigenvalue to said first projected data matrix, said eigenvalue determined from said at least one loading vector, and combining the resulting values for each profile element of each microbiota profile in the first projected data matrix and comparing the combined values for each microbiota profile with a normobiotic to dysbiotic threshold value determined from the corresponding analysis of said plurality of microorganisms or groups of microorganisms in a corresponding GI tract sample from a plurality of normal subjects and/or subjects with dysbiosis,
   wherein step (v) may be performed before or after or concurrently with either of steps (iii) or (iv), and wherein a microbiota profile with said combined differences or said combined resulting values in excess of said respective normobiotic to dysbiotic thresholds is indicative of a likelihood of dysbiosis in the GI tract of the subject from which it has been obtained.

In another particular example of these embodiments there is provided a method for determining the likelihood of GI tract dysbiosis in a plurality of subjects, said method comprising:
(i) providing a plurality of microbiota profiles, wherein each of said microbiota profiles is a profile of the relative levels of a plurality of microorganisms or groups of microorganisms in corresponding samples from the GI tract of said subjects which have been prepared in essentially the same way and wherein each level of each microorganism or group of microorganisms is a profile element, and arranging said microbiota profiles in a test data matrix, wherein each distinct profile element is aligned across the plurality of microbiota profiles,
   and performing steps (ii) to (v) of the method of the invention described supra, wherein step (v) may be performed before or after or concurrently with either of steps (iii) or (iv), and wherein a microbiota profile with said combined differences or said combined resulting values in excess of said respective normobiotic to dysbiotic thresholds is indicative of a likelihood of dysbiosis in the GI tract of the subject from which it has been obtained.

The following sections describe the detail of the method of the invention in terms of the analysis of a single microbiota profile. These details apply mutatis mutandis to the above-described embodiments in which a plurality of microbiota profile are analysed together.

Expressed differently the method of the invention may be considered a method for detecting, diagnosing or monitoring GI tract dysbiosis in a subject wherein a microbiota profile with said combined differences of step (iv) or said combined resulting values of step (v), typically both, in excess of said respective normobiotic to dysbiotic thresholds indicates dysbiosis.

Dysbiosis is defined herein as a microbiota profile that differs or deviates from the microbiota profile that is typical of a normal, healthy, subject, which may be referred to herein as “normobiosis” or a “normobiotic state”. The extent of dysbiosis is a measure of how different a microbiota profile is from a normal microbiota profile or by how much a microbiota profile deviates from a normal microbiota profile. In the context of the diagnosis, monitoring and/or characterisation of diseases and conditions associated with perturbations in the microbiota of the GI tract or the assessment of the risk of developing a disease or condition which has been determined to be associated with a perturbation of the microbiota profile, dysbiosis may be more specifically defined as a microbiota profile that differs from the microbiota profile that is typical of a subject which does not have said disease or condition or is not at risk of developing said disease or condition. A typical microbiota profile may be obtained from a single subject or even a single sample from a single subject, but preferably will be obtained from a plurality of subjects.

A microbiota profile (profile of the relative levels of a plurality of microorganisms or groups of microorganisms) in accordance with the methods of the invention is a numerical representation of such levels that has been obtained from an analysis of a GI tract sample from the subject. The individual values for such levels (the individual profile elements) may be qualitative, quantitative or semi-quantitative, preferably quantitative. The term “amount” could be used in place of “levels” if appropriate.

The profiling of the GI tract sample may involve any convenient means by which the levels of microorganism or group of microorganisms may be measured, preferably quantified. Preferably the means used to prepare the microbiota profiles from a plurality of normal subjects and/or a plurality of subjects with dysbiosis from which the latent variables, e.g. orthogonal latent variables, of step (ii) and the threshold values of steps (iv) and (v) are determined are essentially the same as those used to prepare the at least one microbiota profile of the test data set, although in other embodiments the means may differ. Should different means be used an adjustment vector may need to be calculated and applied in order to permit comparison.

The profiling methods of use in accordance the invention are typically in vitro methods performed using any sample taken from the GI tract.

The GI tract, also referred to as the digestive tract or alimentary canal (and which terms may be used interchangeably with GI tract) is the continuous series of organs beginning at the mouth and ending at the anus. Specifically this sequence consists of the mouth, the pharynx, the oesophagus, the stomach, the duodenum, the small intestine, the large intestine and the anus. These organs can be subdivided into the upper GI tract, consisting of the mouth, pharynx, oesophagus, stomach, and duodenum, and the lower GI tract, consisting of the jejunum, the ileum (together the small intestine), the cecum, the colon, the rectum (together the large intestine) and the anus.

A GI tract sample of use in accordance with the invention may include, but is not limited to any fluid or solid taken from the lumen or surface of the GI tract or any sample of any of the tissues that form the organs of the GI tract. Thus the sample may be any luminal content of the GI tract (e.g. stomach contents, intestinal contents, mucus and faeces/stool, or combinations thereof) as well as samples obtained mechanically from the GI tract e.g. by swab, rinse, aspirate or scrape of a GI tract cavity or surface or by biopsy of a GI tract tissue/organ. Faecal samples are preferred. The sample can also be obtained from part of a GI tract tissue/organ which has been removed surgically. The sample may be a portion of the excised tissue/organ. In embodiments where the sample is a sample of a GI tract tissue/organ the sample may comprise a part of the mucosa, the submucosa, the muscularis externa, the adventitia and/or the serosa of the GI tract tissue/organ. Such tissue samples may be obtained by biopsy during an endoscopic procedure. Preferably the sample is obtained from the lower GI tract, i.e. from the jejunum, the ileum, the cecum, the colon, the rectum or the anus. More preferably the sample is a mucosal or luminal sample. Faecal samples may be collected by the swab, rinse, aspirate or scrape of the rectum or anus or, most simply, the collection of faeces during or after defecation.

The sample may be used in accordance with the invention in the form in which it was initially retrieved. The sample may also have undergone some degree of manipulation, refinement or purification before being used in the methods of the invention. Thus the term “sample” also includes preparations thereof, e.g. relatively pure or partially purified starting materials, such as semi-pure preparations of the above mentioned samples. The term “sample” also includes preparations of the above mentioned samples in which the RNA of which, including the 16S rRNA, has undergone reverse transcription. Further included is the product of the microbial culture of said sample.

The purification may be slight, for instance amounting to no more than the concentration of the solids, or cells, of the sample into a smaller volume or the separation of cells from some or all of the remainder of the sample. Representative cell isolation techniques are described in WO98/51693 and WO01/53525.

In certain embodiments a preparation of the nucleic acid from the above mentioned samples, preferably a preparation in which the nucleic acids have been labelled, is used in accordance with the invention. Such preparations include reverse transcription products and/or amplification products of such samples or nucleic acid preparations thereof. It may be advantageous if the predominant nucleic acid of the nucleic acid preparation is DNA. These preparations include relatively pure or partially purified nucleic acid preparations.

Techniques for the isolation of nucleic acid from samples, including complex samples, are numerous and well known in the art and described at length in the literature. The techniques described in WO98/51693 and WO01/53525 can also be employed to prepare nucleic acids from the above mentioned samples.

The method of the invention may include a step of sample collection and/or sample processing and/or culture, in particular a step of nucleic acid amplification, e.g. genomic nucleic acid amplification, in particular the amplification of nucleic acid carrying nucleotide sequences characteristic of a microorganism or group of microorganisms.

Unless context dictates otherwise, the term “corresponding sample” is used herein to refer to samples of the same type which have been obtained from different subjects and/or at different times in essentially the same way and to which any substantive processing or handling thereof has taken place in essentially the same way.

Methods for profiling microbiota in a GI tract sample include but are not limited to nucleic acid analysis (e.g. nucleic acid sequencing approaches, oligonucleotide hybridisation probe based approaches, primer based nucleic acid amplification approaches), antibody or other specific affinity ligand based approaches, proteomic and metabolomic approaches. Preferably the analysis of the sample will be by nucleic acid sequence analysis and may take the form of a sequencing technique. The Sanger dideoxynucleotide sequencing method is a well-known and widely used technique for sequencing nucleic acids. However more recently the so-called “next generation” or “second generation” sequencing approaches (in reference to the Sanger dideoxynucleotide method as the “first generation” approach) have become widespread. These newer techniques are characterised by high throughputs, e.g. as a consequence of the use of parallel, e.g. massively parallel sequencing reactions, or through less time-consuming steps. Various high throughput sequencing methods provide single molecule sequencing and employ techniques such as pyrosequencing, reversible terminator sequencing, cleavable probe sequencing by ligation, non-cleavable probe sequencing by ligation, DNA nanoballs, and real-time single molecule sequencing.

Nucleic acid sequence analysis may also preferably take the form of an oligonucleotide hybridisation probe based approach in which the presence of a target nucleotide sequence is confirmed by detecting a specific hybridisation event between a probe and its target. In these approaches the oligonucleotide probe is often provided as part of a wider array, e.g. an immobilised nucleic acid microarray. Preferably, the oligonucleotide probe sets and associated methods of WO2012080754 and WO2011043654 may be used to prepare microbiota profiles in accordance with the present invention.

In certain embodiments the methods of the invention may include steps in which the microbiota of a GI tract sample from the subject is profiled, e.g. by any of the above described techniques.

In certain embodiments the microorganisms or groups of microorganisms of which the relative levels thereof are to be determined are preselected, e.g. are microorganisms or groups of microorganisms which are indicator and/or causative species for the disease or condition of interest. This is however not essential so long as comparison within the method of the invention is made between the same microorganisms.

Thus, the term “microorganism” as used in the context of the invention may be any microbial organism, that is any organism that is microscopic, namely too small to be seen by the naked eye, which may be found in the GI tract. In particular, as used herein, the term includes the organisms typically thought of as microorganisms, particularly bacteria, fungi, archaea, algae and protists. The term thus particularly includes organisms that are typically unicellular, but which may have the capability of organising into simple cooperative colonies or structures such as filaments, hyphae or mycelia (but not true tissues) under certain conditions. The microorganism may be prokaryotic or eukaryotic, and may be from any class, genus or species of microorganism. Examples of prokaryotic microorganisms include, but are not limited to, bacteria, including the mycoplasmas, (e.g. Gram-positive, Gram-negative bacteria or Gram test non-responsive bacteria) and archaeobacteria. Eukaryotic microorganisms include fungi, algae and others that are, or have been, classified in the taxonomic kingdom Protista or regarded as protists, and include, but are not limited to, for example, protozoa, diatoms, protophyta, and fungus-like moulds.

In preferred embodiments the groups of microorganisms may be selected from Actinobacteria (e.g. Atopobium, Bifidobacterium), Bacteroidetes (e.g. Bacteroidia, e.g. Alistipes, Bacteroides, Prevotella, Parabacteroides, Bacteroidetes (in particular Bacteroides fragilis), Firmicutes (e.g. Bacilli, e.g. Bacillus, Lactobacillus, Pedicoccus, Streptococcus; Clostridia, e.g. Anaerotruncus, Blautia, Clostridium, Desulfitispora, Dorea, Eubacterium, Faecalibacterium, Ruminococcus; Erysipelotrichia, e.g. Catenibacterium, Coprobacillus, Unclassified Erysipelotrichaceae; Negativicutes, e.g. Dialister, Megasphaera, Phascolarctobacterium; Epsilonproteobacteria; Veillonella/Helicobacter (in particular Dialister invisus, Faecalibacterium prausnitzii, Ruminococcus albus, Ruminococcus bromii, Ruminococcus gnavus, Streptococcus sanguinis, Streptococcus thermophilus)), Proteobacteria (e.g. Gammaproteobacteria, e.g. Acinetobacter, Pseudomonas, Salmonella, Citrobacter, Cronobacter, Enterobacter, Shigella, Escherichia), Tenericutes (e.g. Mollicutes, e.g. Mycoplasma (in particular Mycoplasma hominis), and Verrucomicrobia (e.g. Verrucomicrobiae, e.g. Akkermansia (in particular Akkermansia munciphila)).

In the context of IBS, Firmicutes (Bacilli), Proteobacteria (Shigella/Escherichia), Actinobacteria and Ruminococcus gnavus may be important. Similarly, in the context of IBD, Proteobacteria (Shigella/Escherichia), Firmicutes, specifically Faecalibacterium prausnitzii, and Bacteroidetes (Bacteroides and Prevotella) may be important.

Thus, in certain embodiments references to microbiota profiles may be considered references to bacteriota profiles.

The number of microorgansims or groups of microorganisms of which the relative levels thereof are to be determined is not limited and so may be at least 2, 5, 10, 20, 30, 40, 50, 60, 70, 80, 90 or 100. In other embodiments the number may be less than 500, 200, 150, 100, 90, 80, 70, 60 or 50. Any range defined by endpoints of any of these numbers is hereby disclosed.

In certain embodiments the microbiota profiles may be normalised to account for inter-sample variation within each profiling experimental run and/or inter-experimental variation between each profiling experimental run through the use of appropriate controls during or after the analysis of the samples. Further normalisation to allow for batch variations in lab consumables and to correct for background signals may advantageously be performed.

In certain embodiments a centring vector may be applied to each microbiota profile element and/or each microbiota profile, wherein said vector is derived from the mean value for said microbiota profile element or microbiota profile obtained from corresponding samples from a plurality of normal subjects which have been profiled in the same way.

The test data matrix, if used, will typically be arranged such that each sample is presented as a single row and each microorganism or group of microorganisms of which the relative levels thereof have been determined is presented as a single column. The reciprocal of this arrangement may be used.

The term “latent variables” may refer to a subset of variables from within a data set which relate to potentially unknown correlations and trends. The term further includes variables which are determined from the combination of original variables in a data set (e.g. the level of a microorganism or group of microorganisms in a GI tract sample), specifically those that reflect correlations between variables and trends in the data set in a more meaningful way than the original variables. Thus, latent variables are typically derived from the algorithmic decomposition of a data set, e.g. by regression analysis, e.g. partial least squares regression analysis, principle components analysis (PCA), canonical correlation analysis, redundancy analysis, correspondence analysis, and canonical correspondence analysis. The latent variables may be orthogonal or non-orthogonal. Orthogonal latent variables are latent variables which are orientated perpendicular to one another. Preferably the latent variables of use in the invention are orthogonal latent variables.

The latent variables, in particular the orthogonal latent variables, of use in accordance with the invention may be determined by any convenient means, e.g. the partial least squares regression analysis of the levels of said plurality of microorganisms or groups of microorganisms in GI tract samples from a plurality of normal subjects, preferably which have been profiled in the same way. In certain embodiments the orthogonal latent variables are determined by the orthogonal transformation into principle components of the levels of said plurality of microorganisms or groups of microorganisms in GI tract samples from a plurality of normal subjects, preferably which have been profiled in the same way. In preferred embodiments the orthogonal transformation into principle components is by is PCA, canonical correlation analysis, redundancy analysis, correspondence analysis, and canonical correspondence analysis.

In certain embodiments one or more of the latent variables are the loading vector(s) of use in the invention. In other words, once latent variables are determined no further algorithmic manipulation takes place before they are applied to the test data loading vectors.

In certain embodiments at least 2 vectors, e.g. at least 3, 5, 7, 9, 11, 13, 15, 17, 19 or 20 vectors are applied to the data set. In other embodiments the no more than 50 vectors, e.g. no more than 40, 30, 25, 20, or 15 vectors are applied to the data set. Any range defined by endpoints of any of these numbers is hereby disclosed.

In embodiments wherein said loading vectors are determined from orthogonal latent variables, e.g. by PCA, canonical correlation analysis, redundancy analysis, correspondence analysis, and canonical correspondence analysis the number of loading vectors which may be used in accordance with the invention is limited by the number of microorganisms or groups of microorganisms investigated or the number of GI tract samples from the plurality of normal subjects of subjects with dysbiosis which have been profiled, whichever is the fewer: the greater the number of microorganisms or groups of microorganisms investigated and the greater the number of samples used the greater the number of orthogonal latent variables, and hence associated vectors, that may be present and may be selected from.

The determination of the latent variables, e.g. the orthogonal latent variables, and/or the determination of the loading vectors from said variables may be performed as part of the method of the invention, but more typically may be performed separately or the latent variables, e.g. the orthogonal latent variables, and/or the loading vectors may be obtained from other sources.

In certain embodiments the loading vector(s) are applied in the form of a projection matrix.

In preferred embodiments applying the loading vector(s) to the test data set comprises multiplying the profile elements of the data set by the loading vector(s), e.g. in the form of a projection matrix. Thus, this multiplication may be matrix multiplication.

In other embodiments, step (ii) in which a first projected data set is produced further comprises applying at least one eigenvalue determined from said at least one loading vector to the test data set. The eigenvalue may be applied before or after the loading vector or together with the loading vector. Applying said eigenvalue may comprise multiplying the eigenvalue with the profile elements of the test data set before or after application of the loading vector to the test data set. In other embodiments the eigenvalue may be multiplied with the loading vector before application to the test dataset, e.g. multiplication with the profile elements of the test data set. If appropriate in these embodiments, the references to an eigenvalue may include root, exponentiation or logarithm forms thereof. References to multiplication include matrix multiplication. In still further embodiments where a plurality of eigenvalues is applied in step (ii), the eigenvalues are applied in the form of a matrix with the eigenvalues arranged on the main diagonal, but this is not essential.

The combination of differences between corresponding profile elements in step (iv) may be achieved by any convenient means that results in a meaningful informational output from this step. Combination may simply be the sum or multiplication of the differences between each corresponding element. In other embodiments further manipulation may occur, e.g. the application of root, exponentiation or logarithm techniques to each element and/or each difference between each corresponding element. In still further embodiments the combined differences may also be manipulated further prior to comparison with the normobiotic to dysbiotic threshold values.

In preferred embodiments the combination of each difference between corresponding elements comprises calculating the square of each said difference and then the squared values are summed. In such embodiments step (iii) and step (iv) prior to comparison with normobiotic to dysbiotic threshold values can be expressed as the calculation of Q-residuals for each test microbiota profile.

In preferred embodiments applying the at least one eigenvalue to said first projected data set in step (v) comprises multiplying the profile elements of the first projected data set by the eigenvalue. If appropriate in these embodiments, the references to an eigenvalue may include root, exponentiation or logarithm forms thereof. References to multiplication include matrix multiplication. In still further embodiments where a plurality of eigenvalues is applied in step (v), the eigenvalues are applied in the form of a matrix with the eigenvalues arranged on the main diagonal, although this is not essential. In embodiments in which an eigenvalue is applied in step (ii), the same eigenvalue may be applied in step (v), although this is not essential.

The combination of resulting values in step (v) may be achieved by any convenient means that results in a meaningful informational output from this step. Combination may simply be the sum or multiplication of the resulting values for each profile element. In other embodiments further manipulation may occur, e.g. the application of root, exponentiation or logarithm techniques to each resulting value for each element. In still further embodiments the combined resulting values may also be manipulated further prior to comparison with the threshold values.

In preferred embodiments the combination of each resulting value comprises calculating the square of each resulting value and then the squared values are summed. In such embodiments step (v) prior to comparison with threshold values can be expressed as the calculation of Hotelling's T² for each test microbiota profile from the variance explained by the latent variables of step (ii).

Thus in a preferred embodiment the invention provides a method for determining the likelihood of GI tract dysbiosis in a subject, said method comprising:
(i) providing a test data set, wherein said test data set comprises at least one microbiota profile, said microbiota profile being a profile of the relative levels of a plurality of microorganisms or groups of microorganisms in a sample from the GI tract of the subject and wherein each level of each microorganism or group of microorganisms is a profile element of said test data set,
(ii) applying to said test data set at least one loading vector determined from latent variables within the profiles of the levels of said plurality of microorganisms or groups of microorganisms in corresponding GI tract samples from a plurality of normal subjects, thereby producing a first projected data set,
(iii) providing said first projected data set,
(iv) from said first projected data set calculating the Q-residual of the microbiota profile and comparing the Q-residual of the microbiota profile with a normobiotic to dysbiotic threshold Q-residual value determined from the corresponding analysis of said plurality of microorganisms or groups of microorganisms in corresponding GI tract samples from a plurality of normal subjects and/or subjects with dysbiosis,
(v) from said first projected data set calculating the Hotelling's T² for the microbiota profile from the variance explained by the latent variables of step (ii) and comparing said Hotelling's T² for the microbiota profile with a normobiotic to dysbiotic threshold Hotelling's T² value determined from the corresponding analysis of said plurality of microorganisms or groups of microorganisms in corresponding GI tract samples from a plurality of normal subjects and/or subjects with dysbiosis,
   wherein step (v) may be performed before or after or concurrently with step (iv), and wherein a microbiota profile with a Q-residual or Hotelling's T² in excess of said respective thresholds is indicative of a likelihood of dysbiosis.

The normobiotic to dysbiotic threshold values to which the combination of differences between corresponding profile elements in step (iv) and the combination of resulting values in step (v) are compared are determined from the corresponding analysis of the same plurality of microorganisms or groups of microorganisms in a corresponding GI tract sample from a plurality of normal subjects and/or subjects with dysbiosis. The values displayed by these subjects will provide an indication of where the threshold between normobiosis and dysbisosis lies. The greater the number of subjects analysed the more accurately the threshold may be determined and preferably a plurality of both normal subjects and subjects with dysbiosis will be analysed in the determination of said normobiotic to dysbiotic threshold values. These thresholds represent the boundary between normobiosis and dysbiosis for a particular plurality of microorganisms or groups of microorganisms in a particular GI tract sample which have been profiled in a particular way and so will differ for each overall embodiment of invention and so must be prepared prior to the time at which the comparison with test samples is made.

Corresponding analysis means that the threshold values are determined using essentially the same means to process the profiling results from the corresponding GI tract sample as those used to prepare the combination of differences between corresponding profile elements in step (iv) and the combination of resulting values in step (v). Preferably corresponding analysis further means that the threshold values are determined using essentially the same profiling means on said corresponding samples.

More specifically said threshold values may be determined by:
(i) providing a normobiotic data set, wherein said normobiotic data set comprises at least one microbiota profile, said microbiota profile being a profile of the relative levels of a plurality of microorganisms or groups of microorganisms in a sample from the GI tract of a normal subject and wherein each level of each microorganism or group of microorganisms is a profile element of said data set,
(ii) applying to said first normobiotic data set the same at least one loading vector determined from latent variables within the profiles of the levels of said plurality of microorganisms or groups of microorganisms in corresponding GI tract samples from a plurality of normal subjects as applied to said test data set, thereby producing a first projected normobiotic data set,
(iii) applying to said first projected normobiotic data set a transposed version of said at least one loading vector, thereby producing a second projected normobiotic data set,
(iv) comparing said normobiotic data set with said second projected normobiotic data set and combining the differences between the corresponding profile elements of the second projected normobiotic data set and the normobiotic data set,
(v) applying at least one eigenvalue to said first projected normobiotic data set, said eigenvalue determined from said at least one loading vector, and combining the resulting values for each profile element,

wherein step (v) may be performed before or after or concurrently with either of steps (iii) or (iv), and wherein said combined differences and said combined resulting values are, or at least may contribute to, said respective normobiotic to dysbiotic thresholds.

More specifically said threshold values may also be determined by:
(i) providing a dysbiotic data set, wherein said dysbiotic data set comprises at least one microbiota profile, said microbiota profile being a profile of the relative levels of a plurality of microorganisms or groups of microorganisms in a sample from the GI tract of a subject with dysbiosis and wherein each level of each microorganism or group of microorganisms is a profile element of said data set,
(ii) applying to said first dysbiotic data set the same at least one loading vector determined from latent variables within the profiles of the levels of said plurality of microorganisms or groups of microorganisms in corresponding GI tract samples from a plurality of normal subjects as applied to said test data set, thereby producing a first projected dysbiotic data set,
(iii) applying to said first projected dysbiotic data set a transposed version of said at least one loading vector, thereby producing a second projected dysbiotic data set,
(iv) comparing said dysbiotic data set with said second projected dysbiotic data set and combining the differences between the corresponding profile elements of the second projected dysbiotic data set and the dysbiotic data set,
(v) applying at least one eigenvalue to said first projected dysbiotic data set, said eigenvalue determined from said at least one loading vector, and combining the resulting values for each profile element,

wherein step (v) may be performed before or after or concurrently with either of steps (iii) or (iv), and wherein said combined differences and said combined resulting values are, or at least may contribute to, said respective normobiotic to dysbiotic thresholds.

Typically normobiotic to dysbiotic threshold values are selected to optimise class separation. Expressed differently, threshold values will be set such that values from at least 85%, e.g. at least 90%, 95%, 98% or 99% of the normal subjects analysed will lie on one side of the threshold and values from 85%, e.g. at least 90%, 95%, 98% or 99% of the subjects with dysbiosis that are analysed will lie on the other side of the threshold.

In embodiments in which the test data is quantitative or semi-quantitative the method of the invention may be performed in a way that provides a quantitative or semi-quantitative measure of the extent of dysbiosis or the extent of deviation from normobiosis. Such measures may be advantageous in the context of the diagnosis, prognosis and/or characterisation of diseases or conditions associated with dysbiosis since a greater extent of dysbiosis may indicate a more severe manifestation of the disease or condition or a particular subtype thereof. Such measures may also permit more accurate monitoring of disease progression by offering more comparative data.

In such embodiments the extent to which the combination of differences between corresponding profile elements in step (iv) and the combination of resulting values in step (v) differ from the normobiotic to dysbiotic threshold values to which they are compared will indicate (or provide a measure of, or be proportional to) the extent of dysbiosis.

In more specific embodiments dysbiosis may be quantified by combining the combination of differences between corresponding profile elements in step (iv) (e.g. Q-residuals) and the combination of resulting values in step (v) (e.g. Hotelling's T²) into a single metric for dysbiosis. This second combination may be achieved by any convenient means that results in a meaningful informational output from this step. The second combination may simply be the sum or multiplication of the combination of differences between corresponding profile elements in step (iv) (e.g. Q-residuals) and the combination of resulting values in step (v) (e.g. Hotelling's T²).

Thus the invention provides a method for quantifying dysbiosis, said method comprising performing the above described method of the invention, wherein said comparisons with normobiotic to dysbiotic thresholds together comprise combining the combination of differences between corresponding profile elements in step (iv) and the combination of resulting values in step (v) into a single metric for dysbiosis.

In other embodiments further manipulation may occur, e.g. the application of root, exponentiation or logarithm techniques to the combination of differences between corresponding profile elements in step (iv) (e.g. Q-residuals) and/or the combination of resulting values in step (v) (e.g. Hotelling's T²). In further embodiments weightings may be applied to one or both of these combinations (discussed in more detail below). In still further embodiments the second combination may also be manipulated further. In still further embodiments the Euclidean distance from the origin for both the combination of differences between corresponding profile elements in step (iv) (e.g. Q-residuals) and the combination of resulting values in step (v) (e.g. Hotelling's T²) is calculated. Thus, in certain embodiments the combination of differences between corresponding profile elements in step (iv) (e.g. Q-residuals) and the combination of resulting values in step (v) (e.g. Hotelling's T²) are both squared, then summed and then the square root of that calculation is determined to give said single metric for dysbiosis. This may be represented as the following formula (Formula I) wherein Qres represents the combination of differences between corresponding profile elements in step (iv) (e.g. Q-residuals) and T² represents the combination of resulting values in step (v) (e.g. Hotelling's T²).

$$r = \sqrt{{\{ {T}^{2} \}}^{2} + {Qres}^{2}} \qquad Formula I$$

A single metric is highly convenient and offers advantages in the interpretation of results from different subjects. Surprisingly, it has been found that the combination of differences between corresponding profile elements in step (iv) (e.g. Q-residuals) and the combination of resulting values in step (v) (e.g. Hotelling's T²) are potentially correlated and that a high value for one combination can be associated with a high value of the other. It has been recognised that simply summing these values may over-represent the extent of dysbiosis and so the use of Euclidean distance may be advantageous as it reduces the risk of the single metric over-representing the extent of dysbiosis posed by the simple addition of values in the second combination.

A normobiotic to dysbiotic threshold for the same plurality of microorganisms or groups of microorganisms in a corresponding GI tract sample expressed in the same terms as the above described metric may be calculated in the same way from a plurality of normal subjects and/or subjects with dysbiosis and the extent to which the metric for a test sample differs from said threshold will be proportional to the extent of dysbiosis.

In preferred embodiments the combining of the combination of differences between corresponding profile elements in step (iv) (e.g. Q-residuals) and the combination of resulting values in step (v) (e.g. Hotelling's T²) into a single metric for dysbiosis will comprise scaling said combination of differences between corresponding profile elements in step (iv) (e.g. Q-residuals) and the combination of resulting values in step (v) (e.g. Hotelling's T²) to result in values of similar magnitude, e.g. by dividing the combination of differences between corresponding profile elements in step (iv) (e.g. Q-residuals) and the combination of resulting values in step (v) (e.g. Hotelling's T²) by their respective normobiotic to dysbiotic class thresholds (i.e. the normobiotic to dysbiotic thresholds to which comparison is made in steps (iv) and (v) respectively). This may be represented as the following formula (Formula II) wherein q represents combination of differences between corresponding profile elements in step (iv) (e.g. Q-residuals) and T² represents the combination of resulting values in step (v) (e.g. Hotelling's T²).

$$r = \sqrt{{( \frac{q}{{q}_{thres}} )}^{2} + {( \frac{{T}^{2}}{{T}_{thres}^{2}} )}^{2}} \qquad Formula II$$

A scaled single metric is highly convenient and offers advantages in the interpretation of results from different subjects.

In still further embodiments, in order to quantify dysbiosis, said single metric (preferably said scaled single metric) may further be plotted on a finite, preferably continuous, numerical scale from normobiotic to dysbiotic (or vice versa) with class separation (the boundary between normobiosis and dysbiosis) at a predetermined point, preferably a predetermined integer value, on that finite numerical scale which represents, or is, a combination of, preferably the sum of, the normobiotic to dysbiotic class thresholds (similarly scaled if appropriate) of steps (iv) and (v).

The extent to which a said single metric determined for a test sample differs from the class separation point in the direction of the maximum dysbiotic endpoint of the scale is proportional to the extent of dysbiosis. Preferably, the portion of the numerical scale between the class separation point and the maximum dysbiotic endpoint of the scale is subdivided into discrete regions which further quantify dysbiosis. Preferably said regions have boundaries at defined points, e.g. numerical integers.

Thus the invention provides a method for quantifying dysbiosis, said method comprising performing the above described method of the invention and further plotting said single metric for dysbiosis on a finite, preferably continuous, numerical scale with class separation at a predetermined point which represents, or is, a combination of the normobiotic to dysbiotic class thresholds of steps (iv) and (v), similarly scaled if scaling has been applied.

Similarly, the extent to which a said single metric determined for a test sample differs from the class separation point in the direction of the maximum normobiotic endpoint of the scale is inversely proportional to the extent to which the test sample deviates from the model definition of normal. To help visualise this, the portion of the numerical scale between the class separation point and the maximum normobiotic endpoint of the scale may be subdivided into discrete regions which further quantify deviation from the model definition of normal. Preferably said regions have thresholds at defined points, e.g. numerical integers.

For ease of interpretation, the single metric may be reported in terms of the nearest threshold between the various regions of the scale or class separation point.

Plotting said single metric on such a numerical scale, i.e. a scale with a class separation point that represents, or is, a combination of, preferably the sum of, the normobiotic to dysbiotic class threshold values of steps (iv) and (v), ensures both elements of the metric contribute to the finding of the extent of dysbiosis.

As a result of the fact that said single metric is a combination of two different measures (the combination of differences between corresponding profile elements in step (iv) (e.g. Q-residuals) and the combination of resulting values in step (v) (e.g. Hotelling's T²)) the class boundary (normobiosis to dysbiosis) defined by these measures is two dimensional and simply summing the associated normobiotic to dysbiotic class threshold values for both measures to determine the class separation point on said finite numerical scale may not be able to fully resolve the variation between results from normobiotic and dysbiotic samples which may be seen at or close to the class boundary when only one of these measures is beyond its respective threshold value. It may therefore be more accurate, when further plotting the single metric on a finite numerical scale from normobiotic to dysbiotic (or vice versa) with class separation (the boundary between normobiosis and dysbiosis) at a predetermined point, to set the predetermined class separation point differentially depending on whether or not said test sample has at least one of these measures beyond their respective threshold value. In these embodiments, the class separation point for a test sample having at least one of the combination of differences between corresponding profile elements in step (iv) or the combination of resulting values in step (v) above the normobiotic to dysbiotic class threshold values of steps (iv) and (v), respectively, will correspond to, or preferably be, that of one or other of the exceeded normobiotic to dysbiotic class threshold values of steps (iv) or (v). Also in these embodiments the class separation point for a test sample in which neither of the combination of differences between corresponding profile elements in step (iv) or the combination of resulting values in step (v) are beyond the normobiotic to dysbiotic threshold values of steps (iv) and (v), respectively, will correspond to, or preferably be, the sum of the normobiotic to dysbiotic class thresholds of steps (iv) and (v). In these embodiments “corresponds to” includes root, exponentiation or logarithm forms thereof, preferably the square root form of said threshold values.

Thus the invention provides a method for quantifying dysbiosis, said method comprising performing the above described method of the invention and further plotting the single metric on a finite numerical scale with class separation at a predetermined point, wherein for a test sample having at least one of the combination of differences between corresponding profile elements in step (iv) or the combination of resulting values in step (v) above the normobiotic to dysbiotic class threshold values of steps (iv) and (v), respectively, said class separation point will correspond to that of one or other of the exceeded normobiotic to dysbiotic class threshold values of steps (iv) or (v) and for a test sample in which neither of the combination of differences between corresponding profile elements in step (iv) or the combination of resulting values in step (v) are beyond the normobiotic to dysbiotic threshold values of steps (iv) and (v), respectively, said class separation point will correspond to the sum of the normobiotic to dysbiotic class thresholds of steps (iv) and (v).

In these embodiments it may be convenient if a representative value is applied to the class separation point which remains consistent regardless of the true value of the class separation point applicable to the test sample in question, thus facilitating the use of the same scale to report results from both normobiotic or dysbiotic subjects.

In embodiments in which a portion of the numerical scale between the class separation point and the maximum dysbiotic endpoint of the scale is subdivided into discrete regions which further quantify dysbiosis in terms of defined numerical integers, it may be advantageous to calculate where said single metric falls more precisely as a decimal value between said integer values, thereby a more accurate quantification of dysbiosis may be achieved. In such embodiments calculation of said decimal values between said integers is done via a density or probability distribution function. Numerous techniques are available to the skilled person who would be able to select from or combine such techniques to meet his/her needs or to design new techniques. By way of non-limiting examples the following distributions may be used: lognormal distribution, continuous uniform distribution, discrete uniform distribution, normal (or Gaussian) distribution, student's t-distribution, chi-squared distribution, F-distribution, logit normal distribution, log-logistic distribution, Pareto distribution, Bernoulli distribution, binomial distribution, geometric distribution, Poisson distribution, exponential distribution, gamma distribution, beta distribution. In these embodiments the single metric may still be reported in terms of the nearest threshold between the various regions of the scale or class separation point but, by calculating the decimal value, determining which threshold is nearest will be more accurate.

In preferred embodiments, the effects of technical variation on the plotting of test data onto said finite numerical scale, in particular between said class separation point and said maximum dysbiosis endpoint, is minimised by applying weightings to the combination of differences between corresponding profile elements in step (iv) (e.g. Q-residuals) and/or the combination of resulting values in step (v) (e.g. Hotelling's T²) during the second combination step. Where weightings are applied to both combinations, the weightings may be the same or may differ. One or other may be zero. Suitable weightings may be determined without undue burden by repeatedly analysing a reference sample or a plurality of reference samples, e.g. a sample or samples from a subject with GI tract dysbiosis, in accordance with the invention and the determining what weightings values, if any, for each combination result in the most stable inter-experimental output.

In preferred embodiments, this may be expressed as the following formula (Formula III) wherein q represents combination of differences between corresponding profile elements in step (iv) (e.g. Q-residuals), T² represents the combination of resulting values in step (v) (e.g. Hotelling's T²) and w represents the weighting applied.

$$r = \sqrt{{w}_{q} * {( \frac{q}{{q}_{thres}} )}^{2} + {w}_{{T}^{2}} * {( \frac{{T}^{2}}{{T}_{thres}^{2}} )}^{2}} \qquad Formula III$$

In certain embodiments weightings may be applied to the analysis of samples having at least one of the combination of differences between corresponding profile elements in step (iv) or the combination of resulting values in step (v) in excess of the normobiotic to dysbiotic class threshold values of steps (iv) and (v), respectively, but not to the analysis of a test sample in which neither of the combination of differences between corresponding profile elements in step (iv) or the combination of resulting values in step (v) are beyond the normobiotic to dysbiotic threshold values of steps (iv) and (v), respectively.

In certain embodiments the methods of the invention do not comprise a step in which a microbiota profile from the GI tract sample is compared, e.g. directly, to a corresponding profile representative of a particular disease or condition or stage thereof or to a corresponding profile representative of a healthy subject or a patient with GI tract dysbiosis.

As discussed above, many diseases and conditions, or stages thereof, are believed to be associated with perturbations in the microbiota of the GI tract, or regions thereof. The above described methods of the invention, in particular the quantitative or semi-quantitative methods, may therefore be used to obtain and provide information relevant to the diagnosis, monitoring and/or characterisation of diseases and conditions associated with perturbations in the microbiota of the GI tract or the assessment of the risk of developing a disease or condition which is associated with a perturbation of the microbiota profile of the GI tract. As is clear from the discussion herein, perturbation of the microbiota profile of the GI tract may be considered GI tract dysbiosis.

Thus in a further aspect the invention provides a method for obtaining information relevant to the diagnosis, monitoring and/or characterisation of diseases and conditions associated with perturbations in the microbiota of the GI tract or the assessment of the risk of developing a disease or condition which is associated with by a perturbation of the microbiota profile of the GI tract, said method comprising performing a method as defined above, wherein the product of said method as defined above, the indication of the likelihood of dysbiosis or the extent of dysbiosis, provides said information.

In a further aspect the invention provides a method for diagnosing, monitoring and/or characterising diseases and conditions associated with perturbations in the microbiota of the GI tract or the assessing of the risk of developing a disease or condition which is associated with a perturbation of the microbiota profile of the GI tract, said method comprising performing a method as defined above wherein the indication of the likelihood of dysbiosis or the extent of dysbiosis is indicative of the presence or absence, the risk of developing, the progress of, or the characteristics of said disease or condition associated with perturbations in the microbiota of the GI tract. In these embodiments the method may further comprise a step of making a diagnosis, of monitoring and/or of making a characterisation of a disease or condition associated with perturbations in the microbiota of the GI tract or of making an assessment of the risk of developing a disease or condition which is associated with a perturbation of the microbiota profile of the GI tract based on the indication of the likelihood or the extent of dysbiosis provided in the preceding steps. The results of such a latter step may be recorded and optionally stored on a suitable recording/storage medium and/or communicated to a physician, the subject or intermediary or agent thereof.

In certain embodiments said method is performed with microbiota profiles from a plurality of GI tract samples taken from the patient at different time points. In this way changes in the subjects GI tract microbiota over time may be investigated.

Diseases and conditions associated with perturbations in the microbiota of the GI tract, i.e. dysbiosis, may be considered to be those which may be caused by, or exacerbated by, a shift in the profile of the microbiota of the GI tract (or regions thereof) those which cause, or result in, the display of a profile of the microbiota of the GI tract tract (or regions thereof) that differs from the normal state or those which may be characterised by or identified by the display of a profile of the microbiota of the GI tract tract (or regions thereof) that differs from the normal state. Examples of such diseases and conditions include, but are not limited to, functional GI tract disorders, e.g. Inflammatory Bowel Disease (IBD), Crohn's Disease (CD), Ulcerative Colitis (UC), Irritable Bowel Syndrome (IBS) and dyspepsia; small bowel bacterial overgrowth syndrome and GI tract cancers (e.g. cancer of the mouth, pharynx, oesophagus, stomach, duodenum, jejunum, ileum, cecum, colon, rectum or anus); breast cancer; ankylosing spondylitis; non-alcoholic steatohepatitis; atopic diseases, e.g. eczema, asthma, atopic dermatitis, allergic conjunctivitis, allergic rhinitis and food allergies; metabolic disorders, e.g. diabetes mellitus (type 1 and type 2), obesity and metabolic syndrome; neurological disorders, e.g. depression, multiple sclerosis, dementia, and Alzheimer's disease; autoimmune disease (e.g. arthritis); malnutrition; chronic fatigue syndrome and autism. In preferred embodiments the methods of the invention are performed in the context of IBS.

“Diagnosis” refers to determination of the presence or existence of a disease or condition or stage thereof in an organism. “Monitoring” refers to establishing the extent of, or possible changes in, a disease or condition, particularly when an individual is known to be suffering from a disease or condition, for example to monitor the effects of treatment or the development of a disease or condition, e.g. to determine the suitability of a treatment, to provide a prognosis, and/or to determine if a patient is in remission or relapse. “Characterising” includes determining the features of a particular disease or condition of a subject, e.g. the extent or severity of the disease or condition or the subtype thereof, including likelihood to respond to particular therapies.

“Assessing the risk of a subject developing a disease or condition” refers to the determination of the chance or the likelihood that the subject will develop the disease or condition. This may be expressed as a numerical probability in some embodiments. The assessment of risk may be by virtue of the extent of dysbiosis determined by the methods of the invention.

“Disease” refers to a state of pathological disturbance relative to normal which may result, for example, from infection or an acquired or congenital genetic imperfection.

A “condition” refers to a state of the mind or body of an organism which has not occurred through a recognised disease, e.g. the presence of an agent in the body such as a toxin, drug or pollutant, or pregnancy.

“Stage thereof” refers to different stages of a disease or condition which may or may not exhibit particular physiological or metabolic changes, but do exhibit changes in the profile of the GI tract microbiota. In some embodiments the observed differences in the profile of GI tract microbiota may lead to a previously unappreciated classification of the progress of a disease or condition.

The subject may be any human or non-human animal subject, but more particularly may be a vertebrate, e.g. a mammal, including livestock and companion animals. Preferably the subject is a human, in which case the term “patient” may be used interchangeably with the term “subject”. The subject may be of any age, e.g. an infant, a child, a juvenile, an adolescent or an adult, preferably an adult. In humans, an adult is considered to be of at least 16 years of age and an infant to be up to 2 years of age. In certain embodiments the subject will be an infant, in others it will be a child or an adult. The subject may have or be suspected of having or be or suspected of being at risk of dysbiosis, or the medical condition in question (e.g. IBS and IBD and its subcategories CD and UC).

A “normal” or “healthy” subject is a subject that is not considered to have the illness or disease or other medical condition, the diagnosis of which is the object of the method in question, or a disease, illness or other medical condition that is similar thereto or shares common features and symptoms, e.g. GI symptoms and features. A “normal” or “healthy” GI tract is a GI tract from such subjects. Alternatively put, a “normal” or “healthy” subject (or GI tract) is a subject/GI tract that does not have GI tract dysbiosis. In other embodiments a normal or healthy subject will be essentially free of serious illness or disease or other medical conditions, or at least is a subject that does not have observable or detectable symptoms of any recognised serious illness or disease. In other embodiments a normal or healthy subject will be free of all illness or disease or other medical conditions, or at least does not have observable or detectable symptoms of any recognised illness or disease. Preferably these references to illness, disease or medical condition is a reference to an illness, disease or medical condition of the GI tract.

The word “corresponding” is used to convey the concept that the subject to which the term is applied is the same as another instance of that subject. Thus the essential features that define that subject are shared by the other subject even though precise details may be unique. Alternative terms could be “matching”, “analogous”, agreeing”, “equivalent” or “same as”.

The methods of the invention may be performed on a computer, system or apparatus carrying a program adapted to perform said methods or at least one of the steps thereof. Thus, the methods of the invention may be computer-implemented methods and the invention further provides a computer, system or apparatus carrying a program adapted to perform the methods of the invention. Typically a processor for executing the software and a storage device for storing the test data and the results of one or more steps of the methods of the invention will be present. The processor and storage device will typically be in communication with one another. In one embodiment, the computer, system or apparatus is in communication with a network, such as the Internet, e.g. for communication with laboratories and clinics that communicate test data and/or receive the output of the methods of the invention. The system or apparatus may be further adapted to perform microbiota profiling, or a step thereof, e.g. the step that results in a microbiota profile of use in accordance with the present invention, preferably in a partial or fully automated manner. The invention further provides a computer readable medium carrying said program and such a program per se. In still further aspects the invention provides Formulae I, II and III and the use thereof in the types of methods described generally herein and the specific methods of the invention recited herein.

The results (final output) from the methods of the invention may be provided on computer or human readable media or communicated by any suitable means, electronic or otherwise, for comprehension and/or further interpretation by a skilled person.

The methods of the invention may be used alone as an alternative to other investigative techniques or in addition to such techniques in order to provide information on the microbiota profiles of a subject, e.g. in the diagnosis etc. of diseases and conditions associated with perturbations in the microbiota of the GI tract, in particular in order to diagnose IBS or to dismiss IBS as an explanation for the symptoms presented by the subject. In the context of the diagnosis of IBS, for example, the methods of the invention may be used as an alternative or additional diagnostic measure to diagnosis using imaging techniques such as Magnetic Resonance Imaging (MRI), ultrasound imaging, nuclear imaging, X-ray imaging or endoscopy or IBD serological markers, e.g. anti-Saccharomyces cerevisiae antibodies (ASCA) and peri-nuclear anti-neutrophil cytoplasmic antibodies (pANCA).

In a further aspect the methods described above may comprise a further step of therapeutically treating said subject in a manner consistent with the diagnosis or prognosis made to alleviate, reduce, remedy or modify at least one symptom or characteristic of the disease or condition associated with perturbations in the microbiota of the GI tract that the subject has (e.g. IBS etc.), e.g. by administering a pharmaceutical composition (which may be considered to include faecal microbotal transplants and microbial cultures) and/or performing a surgical procedure appropriate to treat the disease or condition and/or adjusting the lifestyle of the subject in a manner appropriate to treat the disease or condition. In this regard, the invention can be considered to relate to methods for the therapeutic treatment of diseases or conditions associated with perturbations in the microbiota of the GI tract (e.g. IBS etc.) and for guiding and/or optimising such treatments. This may include treatments to remedy, or at least address in part, the GI tract dysbiosis of a subject or the extent thereof.

The invention will now be described by way of the following non-limiting Examples in which:

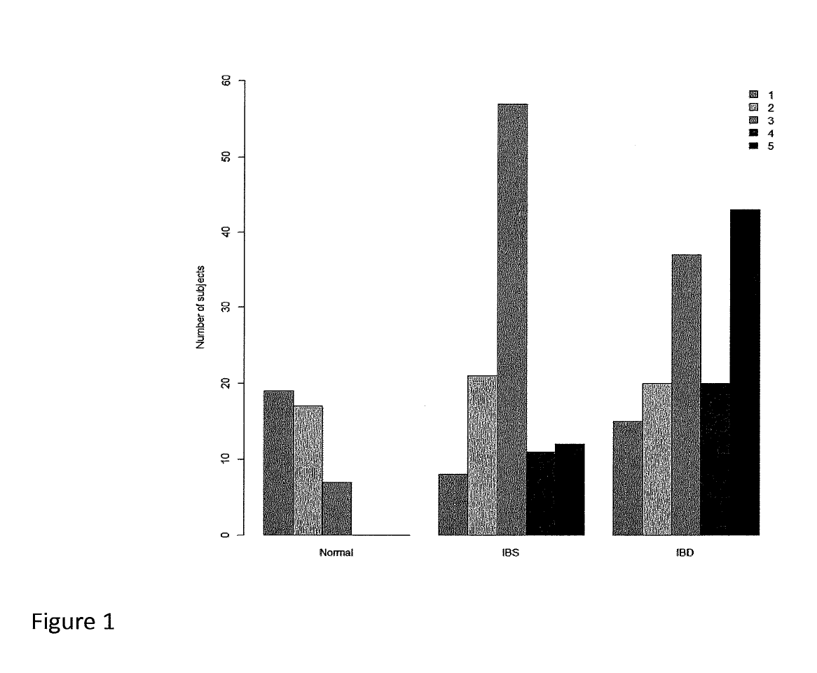

FIG. 1 shows distribution of DI scores 1-5 for the validation cohort as determined in Example 1, showing the increase in DI from normal individuals through IBS patients and finally in IBD patients.

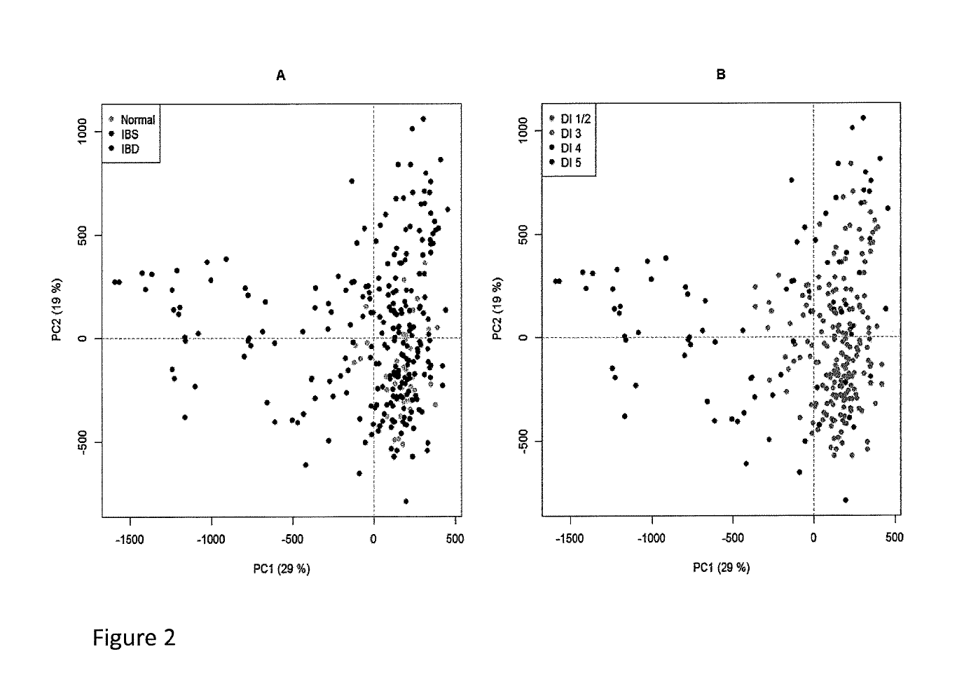

FIG. 2 shows PCA scores for the first two principal components for validation cohort (n=287) based on 54 probes. The two PCs account for 48% of the variation, and points are coloured according to A) cohort: yellow—normal, blue—IBS, and red—IBD; and B) DI: grey=1-2, orange=3, red=4, dark red=5.

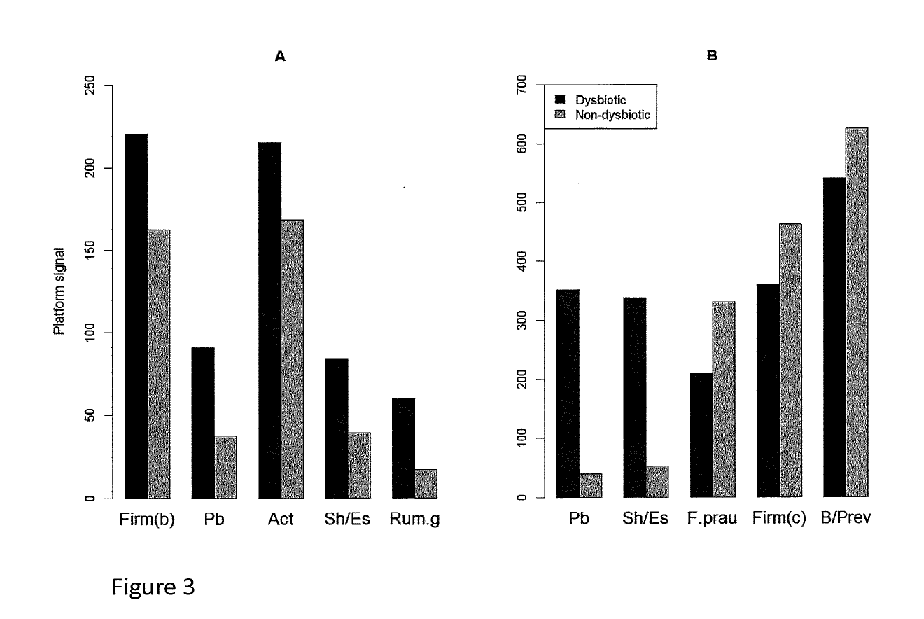

FIG. 3 shows mean normalised signal for top five probes sorted by absolute relative difference between dysbiotic (red) and non-dysbiotic (grey) as determined in Example 1 for A) IBS patients (n=109), and B) IBD patients (n=135). Act; Actinobacteria, B/Prey; Bacteroides/Prevotella, Firm(b); Firmicutes (Bacilli), Firm (c); Firmicutes (Clostridia), F. prau; Faecalibacterium prausnitzii, Pb; Proteobacteria, Rum.g; Ruminococcus gnavus, Sh/Es; Shigella/Escherichia.

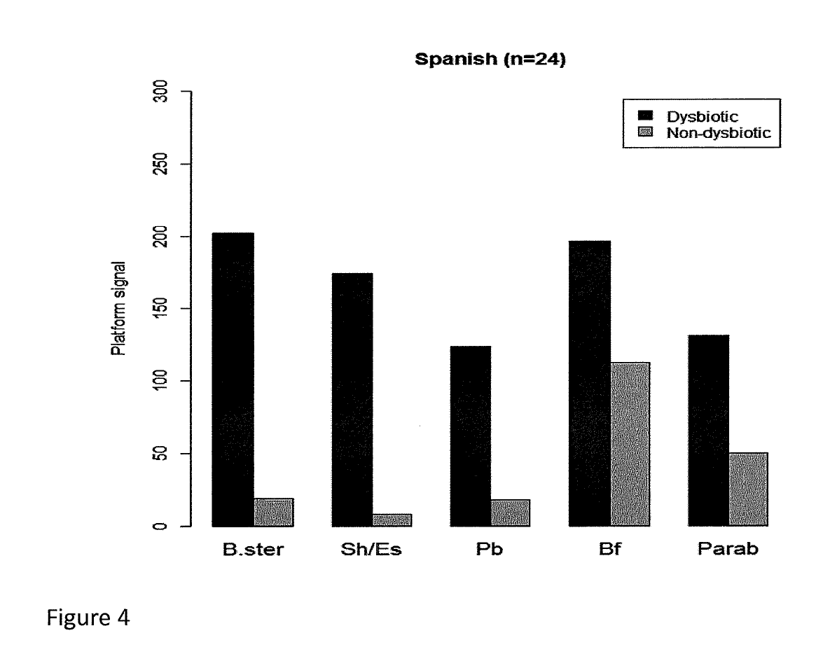

FIG. 4 shows mean normalised signal for probes sorted by absolute relative difference between dysbiotic (red) and non-dysbiotic (grey) as determined in Example 1 for Spanish cohort (n=24). Bf; Bifidobacterium, B.ster; Bacteroides stercoris, Parab; Parabacteroides, Pb; Proteobacteria, Sh/Es; Shigella/Escherichia.

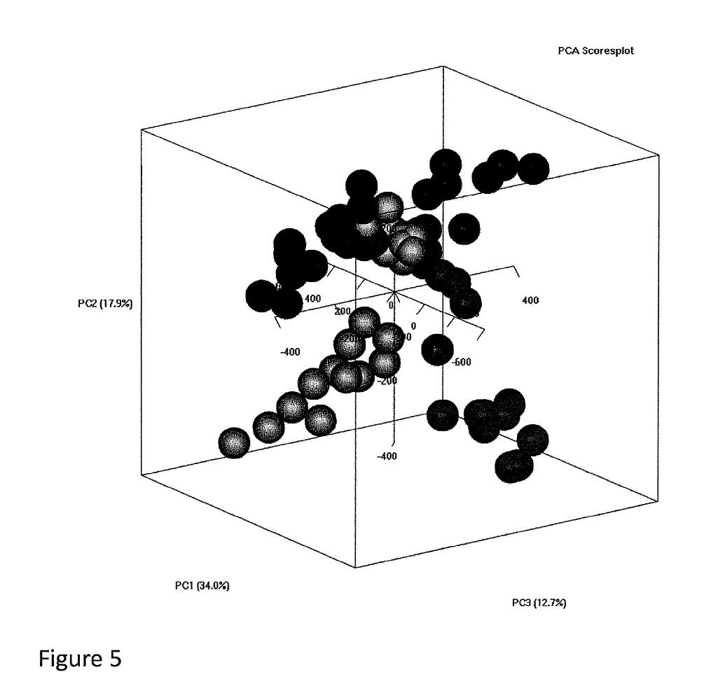

FIG. 5 shows scores for the first three principal components from PCA of normalised data from five healthy subjects collected weekly for up to 14 weeks (n=64). One point is one sample for donor x taken at time point y. The first three PCs account for 65% of the variation, and points are coloured according to donor.

### EXAMPLE 1

### Preparing Profiles of GI Tract Microbiota with 54 Probes Targeting a Plurality Of Microorganisms Or Groups Of Microorganisms and Using Same to Determine Dysbiosis in IBS And IBD Patients

Materials and Methods

Human Samples

Faecal samples were collected from 668 adults (aged 17-76; 69% women), including normal individuals (n=297) and patients with IBS (n=236) and IBD (n=135) (Table 1). Faecal samples were collected from hospitals in Norway, Sweden, Denmark and Spain (72%), as well as from workplaces in Oslo, Norway (28%), in an effort to achieve heterogeneity. The normal donors had no clinical signs, symptoms or history of IBD, IBS or other organic gastrointestinal-related disorders (e.g. colon cancer). The IBS samples were collected as part of prospective studies that used the Rome III diagnostic criteria to identify IBS. The distribution of IBS subtypes was 44% IBS-diarrhoea, 22% IBS-alternating, 17% IBS-constipation, 11% IBS-un-subtyped, and 4% IBS-mixed. The diagnosis of IBD was based on clinical presentation confirmed by colonoscopy. Of the 135 IBD samples, 80 (59%) were treatment-naïve patients and 55 (41%) were IBD patients in remission. The distribution of IBD types was 62% UC and 38% CD for the treatment-naïve group, and 67% UC and 33% CD for the IBD in remission group. Informed consent was obtained for all samples along with approval from local scientific ethics committees.

TABLE 1
Demographic information

Age (years)*

| Categories | Total | Females, % | Mean | Range |
|---|---|---|---|---|
| Normal controls | 297 | 63 | 41 | 21-70 |
| Nordic | 254 | 64 | 42 | 21-70 |
| Danish | 19 | 63 | 42 | 23-61 |
| Spanish | 24 | 50 | 35 | 22-56 |
| IBS^{†} | 236 | 78 | 40 | 17-76 |
| IBS-D | 102 | 79 | 40 | 18-70 |
| IBS-C | 41 | 85 | 42 | 22-73 |
| IBS-M | 10 | 80 | 37 | 19-55 |
| IBS-U | 25 | 88 | 41 | 19-68 |
| IBS-A | 51 | 67 | 39 | 20-62 |
| IBD treatment-naïve | 80 | 56 | 34 | 18-61 |
| CD | 30 | 50 | 33 | 19-53 |
| UC | 50 | 63 | 35 | 18-61 |
| IBD remission^{‡} | 55 | 76 | 42 | 20-69 |
| CD | 18 | 72 | 38 | 20-59 |
| UC | 36 | 78 | 44 | 24-69 |

A, alternating;
C, constipation;
CD, Crohn's disease;
D, diarrhoea;
IBS, irritable bowel syndrome;
IBD, inflammatory bowel disease;
M, mixed;
U, un-subtyped;
UC, ulcerative colitis.
*Precise ages were known for 99% of the total samples used.
^{†}IBS type known for 97% of the total IBS samples used.
^{‡}CD/UC diagnosis known for 99% of the total IBD samples used.

Sample Collection

Samples were collected at home, office or hospital, and frozen within 3-5 days. Faecal samples were mixed with stool transport and recovery buffer (Roche, Basel, Switzerland) in a 1:3 ratio by vortexing. All samples were pulse centrifuged and 600 μl was transferred to a 96-well Lysing Matrix E rack (MP Biomedicals Inc., Santa Ana, Calif., USA). Samples were mechanically lysed twice at 1800 rpm, 40 s on 40 s rest, in a FastPrep-96™ (MP Biomedicals Inc.). Lysed samples were centrifuged (5 min, 1300 g, PlateSpin II centrifuge, Kubota, Tokyo, Japan), and 250 μl was incubated at 65° C. for 15 min with 250 μl lysis buffer BLM and 20 μl protease. A 400 μl aliquot of each protease-treated faecal sample was used to extract total genomic DNA according to mag™ maxi kit instructions (LGC Genomics, Berlin, Germany), adjusted for a MagMAX™ express 96 DNA extraction robot (Life Technologies, Waltham, Mass., USA).

The polymerase chain reaction (PCR) primers (targeted the 16S rRNA gene and were used to amplify 1180 base pair fragments containing seven variable regions (V3-V9). This was followed by a reaction clean-up as described by Vebø et al (Vebo HC, Sekelja M, Nestestog R, et al. Temporal development of the infant gut microbiota in immunoglobulin E-sensitized and nonsensitized children determined by the GA-map infant array. Clin Vaccine Immunol 2011; 18: 1326-1335) with minor modifications.

Sample Analysis: SNE, Hybridisation and Detection

The PCR template (>75 ng) was used in an single-nucleotide extension (SNE) reaction described in Vebø et al, with the following modifications: a final volume of 25 μl containing 0.5 μM BIOTIN-11-ddCTP (Perkin Elmer, Waltham, Mass., USA) was used through five labelling cycles to label a probe-set of 55 probes (0.01 μM) (54 bacteria target probes and Universal control). Complementary probes coupled to carboxyl barcoded magnetic beads (BMBs, Applied BioCode, Santa Fe Springs, Calif., USA) were hybridised to the SNE probes and quantified using a BioCode 1000A Analyzer (Applied BioCode). In brief, a 10 μl SNE sample was added to a 40 μl reaction volume containing 31.2 μl BMB buffer, hybridisation control and 1.8 μl coupled BMBs. Samples were incubated at 700 rpm, 95° C. for 3 min, followed by 700 rpm, 45° C. for 15 min in a Vortemp™ 56 (Labnet International Inc., Edison, N.J., USA). A 25 μl BMB buffer containing 20 μg/ml streptavidin R-phycoerythrin LumiGrade ultrasensitive reagent (Roche) was added to each sample before 90 minutes of incubation at 700 rpm, 45° C. Finally, samples were washed according to Applied BioCode's recommendations. The hybridisation signal was processed by the BioCode 1000A Analyzer software (Applied BioCode). The software identified and quantified median signals, bead count and flags, and raw data files were exported for further analysis.

Probe Identification, Selection, In Silico and In Vitro Testing

To establish and optimise the most applicable bacterial probe set, data from previous IBD and IBS intestinal microbiota research was compiled based on pre-defined search criteria to provide >500 bacterial observations associated with the occurrence of IBD and IBS. From a combined dataset of 496 16S rRNA gene sequences (consensus sequence[s] for each species, chosen from all available long 16S rRNA sequences and purified to avoid sequences errors) from 269 bacterial species, probes were designed to cover major bacterial observations made from the literature. All probes were designed according to Vebø et al with a minimum melting temperature (Tₘ) of 60° C. by the nearest-neighbour method for the target group where the nucleotide 3′ end of the probe is a cytosine; non-target group probe requirements were a Tₘ of 30° C. or absence of a cytosine as the nucleotide adjacent to the 3′ end of the probe. Each probe was designed to target a bacterial species or group, i.e. Faecalibacterium prausnitzii (species), Lactobacillus (genus), Clostridia (class) and Proteobacteria (phylum), based on their 16S rRNA sequence (V3-V9). Probes that satisfied target detection and non-target exclusion in silico were evaluated for cross-labelling, self-labelling and cross hybridisation before final validation was performed against bacterial strains in vitro.

After in vitro testing, a panel of 124 optimal probes was further selected using variable selection methods: variable importance in projection, selectivity ratio and interval partial least squares using data from a selection of normal and IBS samples (data not shown). The variables (probes) were selected based on their ability to distinguish between samples isolated from normal healthy individuals and IBS patients. A final panel of 54 probes was selected covering the sites across V3 to V7 on the 16S rRNA sequence. Bacterial target specificity, tested with the 54-probe set against 368 single bacterial strains was performed to define the target bacteria for each probe. As shown in Table 2, the probes detect bacteria within the six phyla; Firmicutes, Proteobacteria, Bacteroidetes, Actinobacteria, Tenericutes and Verrucomicrobia, covering 11 taxonomic bacterial classes and 36 genera.

TABLE 2
List of the bacterial targets of the 54 labelling probes

| Probe number | Phylum | Class | Genus |
|---|---|---|---|
| 1 | Actinobacteria | Actinobacteria | Atopobium |
| 2 | Actinobacteria | Actinobacteria | Bifidobacterium |
| 3 | Actinobacteria | Actinobacteria | |
| 4 | Actinobacteria | | |
| 5 | Bacteroidetes | Bacteroidia | Alistipes |
| 6 | Bacteroidetes | Bacteroidia | Alistipes |
| 7 | Bacteroidetes | Bacteroidia | Bacteroides |
| 8 | Bacteroidetes | Bacteroidia | Bacteroides/Prevotella |
| 9 | Bacteroidetes | Bacteroidia | Bacteroides |
| 10 | Bacteroidetes | Bacteroidia | Bacteroides |
| 11 | Bacteroidetes | Bacteroidia | Bacteroides |
| 12 | Bacteroidetes | Bacteroidia | Bacteroides |
| 13 | Bacteroidetes | Bacteroidia | Parabacteroides |
| 14 | Bacteroidetes | Bacteroidia | Parabacteroides |
| 15 | Bacteroidetes | Bacteroidia | Prevotella |
| 16 | Firmicutes | Bacilli | Bacillus |
| 17 | Firmicutes | Bacilli | Lactobacillus |
| 18 | Firmicutes | Bacilli | Lactobacillus |
| 19 | Firmicutes | Bacilli | Pedicoccus/Lactobacillus |
| 20 | Firmicutes | Bacilli | Streptococcus |
| 21 | Firmicutes | Bacilli | Streptococcus |
| 22 | Firmicutes | Bacilli | Streptococcus |
| 23 | Firmicutes | Bacilli | Streptococcus |
| 24 | Firmicutes | Bacilli | |
| 25 | Firmicutes | Bacilli/Clostridia | Streptococcus/Eubacterium |
| 26 | Firmicutes | Clostridia | Anaerotruncus |
| 27 | Firmicutes | Clostridia | Blautia |
| 28 | Firmicutes | Clostridia | Clostridium |
| 29 | Firmicutes | Clostridia | Clostridium |
| 30 | Firmicutes | Clostridia | Desulfitispora |
| 31 | Firmicutes | Clostridia | Dorea |
| 32 | Firmicutes | Clostridia | Eubacterium |
| 33 | Firmicutes | Clostridia | Eubacterium |
| 34 | Firmicutes | Clostridia | Eubacterium |
| 35 | Firmicutes | Clostridia | Faecalibacterium |
| 36 | Firmicutes | Clostridia | Ruminococcus |
| 37 | Firmicutes | Clostridia | |
| 38 | Firmicutes | Clostridia | |
| 39 | Firmicutes | Erysipelotrichia | Catenibacterium |
| 40 | Firmicutes | Erysipelotrichia | Coprobacillus |
| 41 | Firmicutes | Erysipelotrichia | Unclassified |
| | | | Erysipelotrichaceae |
| 42 | Firmicutes | Negativicutes | Dialister |
| 43 | Firmicutes | Negativicutes | Megasphaera/Dialister |
| 44 | Firmicutes | Negativicutes | Phascolarctobacterium |
| 45 | Firmicutes | Negativicutes | |
| 46 | Firmicutes | Negativicutes/ | Veillonella/Helicobacter |
| | | Epsilonproteobacteria/ | |
| | | Clostridia | |
| 47 | Firmicutes/ | | |
| | Tenericutes/ | | |
| | Bacteroidetes | | |
| | species | | |
| 48 | Proteobacteria | Gammaproteobacteria | Acinetobacter |
| 49 | Proteobacteria | Gammaproteobacteria | Pseudomonas |
| 50 | Proteobacteria | Gammaproteobacteria | Salmonella, Citrobacter, |
| | | | Cronobacter, Enterobacter |
| 51 | Proteobacteria | Gammaproteobacteria | Shigella/Escherichia |
| 52 | Proteobacteria | | |
| 53 | Tenericutes | Mollicutes | Mycoplasma |
| 54 | Verrucomicrobia | Verrucomicrobiae | Akkermansia |

Data Pre-Processinq

To ensure high quality assurance, several quality control criteria were applied to the detection data for each sample: 1) a bead count >2 for each probe; 2) the hybridisation control (HYC) median signal >13,000; 3) a median background signal <500; and 4) a universal control median signal >4500. Normalisation was applied by first dividing the signal intensity of each probe in each sample by the signal intensity for HYC for that sample, and multiplying by 1000. This was done to adjust for sample differences due to pipetting or hybridisation. Subsequently, normalisation to adjust for run differences was applied by dividing the HYC-normalised signal of each probe in each sample by the median HYC-normalised signal of each probe for replicates of a synthetic DNA control, and multiplying by 1000. Prior to normobiotic microbiota profile calibration, normalised signal intensities below 15 were set to 0 to remove for low background noise and data was mean centred. Test and validation samples were normalised, and normalised signals below 15 were set to 0 before data was mean centred using mean probe signals from the normobiotic reference cohort.

Dvsbiosis Test Development and Validation

Principal component analysis (PCA) was used to build a normobiotic microbiota profile (model). The boundary between non-dysbiotic and dysbiotic was determined by calculating confidence regions for the values of Hotelling's T-square and Q statistics given by PCA scores in the model. Geometrically this corresponds to a rectangle with one corner located at the origin which classifies samples located within the rectangle as non-dysbiotic and samples located outside as dysbiotic. Analysis of T-square and Q statistics scaled by the confidence limit showed that the Euclidian distance from the origin had a log-normal like distribution (data not shown). Euclidian distance from the origin was used to merge the two dimensions, and weighting was performed to capture the effect of T-squared and Q statistics as appropriate. A single numeric representation of the degree of dysbiosis, defined as the Dysbiosis Index (DI), was derived from a log-normal distribution by assigning estimated portions of the distribution to different values on a scale set from 0-5. A DI value of 2 was defined as class separation represented by the identified confidence limits; a DI of 2 or lower being the non-dysbiotic region and a DI of 3 or higher being the dysbiotic region. The higher the DI above 2, the more the sample is considered to deviate from normobiosis, e.g. sample A with DI=4 is farther away from the normobiotic reference cohort in the Euclidian space than sample B with DI=3, thus A is more dysbiotic than B. The scale was optimised with emphasis on reducing technical variation between replicates, meaning that the integer part of the numeric output is decided by predetermined levels of the Euclidian distance. To create the present test, 211 normal individuals were selected and randomly split into a training set (n=165) designed to build models and a test set (n=46) designed to tune parameters. Duplicate samples were run, and mean normalised signal was used for training and testing. Sample demographics for the two groups were similar (Table 3). Additionally, a set of IBS patients were included in the test set (n=127). A number of models were developed and evaluated, and the frequency of dysbiosis in the test set was used as measure of model performance. For the final PCA model, 15 principal components were used, and a 98% confidence limit was determined for T-squared and Q statistics to define class separation. When the model is used to score other samples, values outside these limits are defined as dysbiotic.

TABLE 3
Sample sets used for test development and validation

| | | Age, | | Sample type, n | | |
|---|---|---|---|---|---|---|
| Cohort | Samples, n | mean | Female, % | Normal | IBS | IBD |
| Training | 165 | 42 | 64 | 165 | — | — |
| Test | 173 | 40 | 73 | 46 | 127 | — |
| Validation | 287 | 39 | 71 | 43 | 109 | 135 |
| Full cohort | 625 | 40 | 70 | 254 | 236 | 135 |

External validation using an independent test set comprising normal, IBS and IBD subjects (n=287) was used to assess the clinical diagnostic performance of the model (Table 4). The validation set subjects were all from unique donors who had not been included in the normal reference population used for normobiotic profile calibration or in parameter tuning. Each sample was processed using the finalised algorithm which converts data for each sample into a single integer, i.e. the DI, which represents the degree of dysbiosis based on bacterial abundance and profile within a sample relative to the established normobiotic profile. A DI>2 represents a potentially clinically relevant deviation in microbiotic profile from that of the normobiotic reference population. Finally, the dysbiosis frequency was calculated. Additionally, PCA was performed on the validation set to investigate differences in microbiota profile between the three subject groups.

TABLE 4
Percentage dysbiosis and mean DI score in validation cohort

| | | Dysbiotic, % | |
|---|---|---|---|
| Cohort | Total | (95% CI) | DI, mean |
| Normal controls | 43 | 16 (±11) | 1.72 |
| IBS | 109 | 73 (±8) | 2.98 |
| IBS-D | 34 | 76 (±14) | 3.03 |
| IBS-C | 26 | 73 (±17) | 3.00 |
| IBS-M | 3 | 67 | 3.33 |
| IBS-U | 25 | 72 (±18) | 3.04 |
| IBS-A | 20 | 70 (±20) | 2.85 |
| IBD treatment-naïve | 80 | 70 (±10) | 3.31 |
| CD | 30 | 80 (±14) | 3.60 |
| UC | 50 | 64 (±13) | 3.14 |
| IBD remission | 55 | 80 (±11) | 3.15 |
| CD | 18 | 89 (±14) | 3.65 |
| UC | 36 | 75 (±14) | 2.92 |

A, alternating;
C, constipation;
CD, Crohn's disease;
D, diarrhoea;
DI, Dysbiosis Index;
IBD, inflammatory bowel disease;
IBS, irritable bowel syndrome;
M, mixed;
U, un-subtyped;
UC, ulcerative colitis.

Technical Performance

The EU directive for in vitro diagnostic tests was followed to ensure compliance with a CE-marked test. The main technical parameters evaluated were precision and quantitative range of the test; both at probe signal level and at final output level (i.e., DI). At probe level, precision of signals (coefficient of variation [CV], percentage) varied with raw signal intensity. Signals below 500 IU were regarded as background noise; therefore measurement of variance was not applicable. For signals above 500 IU precision was estimated to be 8.4%, using repeated runs for six donors over six faecal extractions per donor over 2 days (n=328). A CV below 10% was set as a criterion in development of the DI algorithm. Based on repetitive measurements of 139 dysbiotic samples, 94% of the samples showed CVs below 10%. In addition, several in-process test steps were evaluated (data not shown).

Faecal Microbiota Variation Over Time

Variation in microbiota over time was investigated both for normalised data across the selected probe-set, and for the test result (DI). Faecal samples were collected from five donors (aged 24-38; 80% women) at a 1-week interval for up to 14 weeks. PCA of normalised data was performed, and statistical assessment of variation in the signals for donor and sampling time (weekly) was conducted using R package ffmanova, an implementation of fifty-fifty multivariate analysis of variance (ANOVA).

Statistical Analysis

All data were analysed at GA (Genetic Analysis AS, Oslo, Norway). Categorical data were expressed as the number of subjects (and percentage) with a specified condition or clinical variable, and the mean as appropriate. The Mann-Whitney U test was used for testing DI values. All tests were two-sided, and the chosen level of significance was P<0.05. Analysis was done using the statistical computing language R version 3.0.2 and MATLAB 2011b, The MathWorks, Inc., Natick, Mass., United States.

Results

Frequency of Dysbiosis in Normal, IBS and IBD Subjects

Validation of the presently described test was performed by comparing frequency of dysbiosis in a set of 287 samples, including normal individuals previously not included in the normobiotic profile calibration (n=43) and patients with IBS (n=109) and IBD (n=135) (Table 3). The results in the validation cohort are given in Table 4. Of the 43 normal samples included in the validation cohort, seven (16%) were determined as being dysbiotic, with the distribution of DI scores for validation cohort shown in FIG. 1. Among the IBS patients, 80 out of 109 (73%) were determined as being dysbiotic. In the IBD cohort, 100 out of 135 (74%) were determined as being dysbiotic, including 56 out of 80 (70%) treatment-naïve IBD patients, and 44 out of 55 (80%) IBD patients in clinical remission. The distribution of DI between IBS and IBD patients was significantly different (P<0.01). FIG. 1 suggests that the distribution of DI scores for the IBD cohort shows a greater shift towards higher values than IBS. Similarly, within both IBD cohorts, the frequency of dysbiosis for CD (80% and 89%, respectively) was higher than that for UC (64% and 75%), with a significant difference in DI values between CD and UC (P=0.03).

The test was also applied to a set of 43 available samples from normal individuals from Denmark (n=19; aged 23-61; 63% women) and Spain (n=24; aged 22-56; 50% women). Seven of the 19 Danish samples were determined as being dysbiotic with mean DI of 2.16, resulting in 37% dysbiotic (95% CI, 15%-59%). Among the Spanish samples, 10 out of 24 were determined as being dysbiotic with mean DI of 2.58, resulting in 42% dysbiotic (95% confidence interval [CI], 22%-62%). While results for the Danish normal cohort were not significantly different from the normal validation cohort (P>0.05), we observed that 50% ( 5/10) of the dysbiotic samples in the Spanish cohort showed a DI above 3.

Bacterial Profile in Dysbiosis

Applying PCA to the validation cohort using normalised data for all 54 probes demonstrated relative clustering of samples by disease cohorts. The scores for the first two principal components (PC), accounting for 48% of the variance in the data, showed a tighter cluster for normal subjects in the bottom right corner compared with a more diverse spread for subjects with IBD and IBS (FIG. 2A). The sample distribution in the scores plot was found to be linked to the degree of dysbiosis, with a central cluster of non-dysbiotic samples surrounded by samples with ‘weak’ dysbiosis (DI=3), and the samples with the most ‘severe’ dysbiosis (DI=5) scattered outside this cluster (FIG. 2B). Both the first and second principal component each separate the normal samples from IBS and IBD samples to a certain degree. The scatter of DI values implies that different bacteria dominate dysbiosis for different samples. To further investigate which bacterial groups were the main contributors to dysbiosis in IBD and IBS, differences in overall mean normalised signal between dysbiotic and non-dysbiotic status for each of the 54 probes were calculated. The predominant bacteria contributing to dysbiosis within the IBS cohort were Firmicutes (Bacilli), Proteobacteria (Shigella/Escherichia), Actinobacteria and Ruminococcus gnavus (FIG. 3A). Similarly, the predominant bacteria within the IBD cohort were Proteobacteria (Shigella/Escherichia), Firmicutes, specifically Faecalibacterium prausnitzii, and Bacteroidetes (Bacteroides and Prevotella) (FIG. 3B). Interestingly, Proteobacteria (Shigella/Escherichia) was among the top five dysbiosis-contributing bacterial groups for both IBS and IBD, implying similarities in dysbiosis between IBS and IBD. However, all bacterial groups that contributed most to dysbiosis in the IBS cohort showed increased probe signal intensity compared to non-dysbiotic patients, while for the IBD cohort, both reduced (F. prausnitzii) and increased probe signal intensities were the main contributors to dysbiosis.

We found a single probe with a differential signal between samples from the Spanish and Scandinavian cohorts (P<0.01; Benjamini-Hochberg correction). The probe targets Firmicutes (Streptococcus), and this signal was found to be elevated in the Spanish samples compared to the Scandinavian cohort. FIG. 4 shows the predominant bacteria contributing to dysbiosis within the Spanish samples. As expected, Proteobacteria (Shigella/Escherichia) is again found to be a contributing bacteria in dysbiosis. Additionally, Bacteroides stercoris and Bifidobacterium contribute to dysbiosis, which potentially could be linked to differences in e.g. diet between Scandinavian countries and the Mediterranean region.

Faecal Microbiota Variation Over Time

Faecal samples were collected from five individuals at 1-week intervals for up to 14 weeks. PCA of the normalised data (n=64) revealed that most variability in the longitudinal faecal microbiota analysis was related to inter-individual variability; donors could clearly be distinguished by the three first and most important PCs in the score plot (FIG. 5). In this study, samples were clustered according to faecal donor independently of sample collection time. The three first PCs described 65% of the total variability in the faecal microbiota data.

The significance of the PCs was analysed by ffmanova using normalised data and only the main effects of donor and sampling time (weekly) were included in the model. The results show that the average amount of variation between donors was greater than that within a donor (P<0.001) with explained variances based on sums of squares of 0.48. The variation between sampling time was not significant (P=0.26), with explained variances based on sums of squares of 0.11. The low level of variation within one individual over time is crucial in utilising the test for monitoring changes during treatment for the purpose of altering the microbiota profile.

Discussion

In this Example, we demonstrate the performance of a novel gut microbiota test, aiming to identify and characterise dysbiosis by determining deviation from normobiosis. Such a diagnostic approach contrasts to direct diagnosis of a particular disease. Characteristic sets of bacteria are required in a healthy normobiotic gut microbiota, and deviation will represent a dysbiotic state. Quantitative measurement of deviation in bacterial microbiota makes it possible to characterise dysbiosis in samples from IBS and IBD patients based on a single diagnostic algorithm targeting normobiosis.

The present test is a broad-spectrum, reproducible, precise, high-throughput, easy to use method of quantifying the extent of dysbiosis that is especially suitable for clinical use. This test gives an algorithmically-derived DI based on bacterial abundance and profile within a sample. This DI is an indicator of the degree to which an individual's microbiome deviates from that of a normal reference population and could potentially be highly relevant in clinical diagnosis and monitoring of the progression of conditions such as IBD and IBS. The stability of the human gut microbiota is another important feature if microbial characterisation is to play a role in diagnosis, treatment, and prevention of disease. It has been shown that, in an individual's microbiota, 60% of the bacterial strains persisted over the course of 5 years. In our corresponding study, we found only a low within-individual variation in weekly sampling over 14 weeks.

The presently described test has been used to detect high frequency of dysbiosis in IBS and IBD patients and low frequency in normal individuals. Both IBD patients in remission and treatment-naïve IBD patients reported DI scores well above the threshold of 2 with a dysbiosis frequency of 80% and 70%, respectively. Rome III-diagnosed IBS patients showed a frequency of 73%, confirming previous observations, while the frequency in normal individuals was 16%.

Dysbiosis is associated with many diseases, including IBS, different forms of IBD, obesity and diabetes, and has also been implicated in depression and autism. In recent years, new treatment options have emerged with respect to restoring the balance of the microbiota in dysbiotic patients. FMT is now regarded as the most effective treatment in relapsing Clostridium difficile colitis and is currently being studied in phase I to IV clinical trials in many of the aforementioned conditions (CD, phase II/III NCT01793831; UC, phase I NCT01947101, phase II NCT01896635, phase II/III NCT01790061; IBD including CD and UC, phase IV NCT02033408).

A key barrier in the interpretation of FMT data has been the variability in bacterial composition of donor microbiota, not only related to pathogenic organisms but also to the composition of the normally occurring microflora, further highlighting the importance of identifying a method to sufficiently characterise both pathogenic and non-pathogenic microbes. The ability to characterise an individual's microbiome and monitor alterations may allow for the prediction of therapeutic outcome or even relapse in such conditions. It may also help to explain why a patient is refractory to particular therapeutic regimens and aid adaptation of the regimen accordingly. Furthermore, rapid and reproducible detailed bacterial profiles from normobiotic and dysbiotic individuals may aid the continuation of innovative therapeutic approaches such as FMT. Thus, use of the test could prove clinically useful in determining dysbiosis, not only in IBS and IBD patients, but also in other conditions where knowledge about the microbiota profile might prove clinically useful, in the subsequent monitoring of prescribed treatment regimens, and in the evolution of new therapeutic approaches.

### EXAMPLE 2

### Representative Analysis of a Sample Using 54 Probes Targeting a Plurality of Microorganisms or Groups of Microorganisms

TABLE 5
Measured signals for 54 test probes and 4 control probes (bold)

| | Probe | | | | | | | | | |
|---|---|---|---|---|---|---|---|---|---|---|
| | 0 | 1 | 2 | 3 | 4 | 5 | 6 | 7 | 8 | 9 |
| Measured | 1187 | 41 | 4411 | 2198 | 9 | 10 | 89 | 38 | 50 | 1691 |
| signal | | | | | | | | | | |
| | Probe | | | | | | | | | |
| | 10 | 11 | 12 | 13 | 14 | 15 | 16 | 17 | 18 | 19 |
| Measured | 1358 | 27 | 60 | 303 | 4330 | 26 | 26 | 250 | 44 | 885 |
| signal | | | | | | | | | | |
| | Probe | | | | | | | | | |
| | 20 | 21 | 22 | 23 | 24 | 25 | 26 | 27 | 28 | 29 |
| Measured | 1369 | 7 | 68 | 65 | 49 | 24 | 10 | 32 | 45 | 67 |
| signal | | | | | | | | | | |
| | Probe | | | | | | | | | |
| | 30 | 31 | 32 | 33 | 34 | 35 | 36 | 37 | 38 | 39 |
| Measured | 2676 | 798 | 17 | 2 | 2 | 528 | 32 | 203 | 1 | 529 |
| signal | | | | | | | | | | |
| | Probe | | | | | | | | | |
| | 40 | 41 | 42 | 43 | 44 | 45 | 46 | 47 | 48 | 49 |
| Measured | 765 | 38 | 97 | 183 | 120 | 8 | 5068 | 4548 | 4 | 47 |
| signal | | | | | | | | | | |

| | Probe | | | | | | | |
|---|---|---|---|---|---|---|---|---|
| | 50 | 103 | 104 | 105 | 106 | 107 | 126 | 127 |
| Measured | 1 | 155 | 419 | 11 | 81 | 19.352 | 4 | 6 |
| signal | | | | | | | | |

TABLE 6
Measured signals adjusted by hybridisation control probe (107) - test values divided by value of 107 probe.

| | Probe | | | | | | | | | |
|---|---|---|---|---|---|---|---|---|---|---|
| | 0 | 1 | 2 | 3 | 4 | 5 | 6 | 7 | 8 | 9 |
| Hyb | 61.33732947 | 2.118644068 | 227.9350971 | 113.5799917 | 0.46506821 | 0.516742456 | 4.599007854 | 1.963621331 | 2.583712278 | 87.38114924 |
| ad- | | | | | | | | | | |
| justed | | | | | | | | | | |
| data | | | | | | | | | | |
| | Probe | | | | | | | | | |
| | 10 | 11 | 12 | 13 | 14 | 15 | 16 | 17 | 18 | 19 |
| Hyb | 70.17362547 | 1.39520463 | 3.100454733 | 15.6572964 | 223.7494833 | 1.343530384 | 1.343530384 | 12.91856139 | 2.273666804 | 45.73170732 |
| ad- | | | | | | | | | | |
| justed | | | | | | | | | | |
| data | | | | | | | | | | |
| | Probe | | | | | | | | | |
| | 20 | 21 | 22 | 23 | 24 | 25 | 26 | 27 | 28 | 29 |
| Hyb | 70.74204217 | 0.361719719 | 3.513848698 | 3.358825961 | 2.532038032 | 1.240181893 | 0.516742456 | 1.653575858 | 2.32534105 | 3.462174452 |
| ad- | | | | | | | | | | |
| justed | | | | | | | | | | |
| data | | | | | | | | | | |
| | Probe | | | | | | | | | |
| | 30 | 31 | 32 | 33 | 34 | 35 | 36 | 37 | 38 | 39 |
| Hyb | 138.2802811 | 41.23604795 | 0.878462174 | 0.103348491 | 0.103348491 | 27.28400165 | 1.653575858 | 10.48987185 | 0.051674246 | 27.3356759 |
| ad- | | | | | | | | | | |
| justed | | | | | | | | | | |
| data | | | | | | | | | | |
| | Probe | | | | | | | | | |
| | 40 | 41 | 42 | 43 | 44 | 45 | 46 | 47 | 48 | 49 |
| Hyb | 39.53079785 | 1.963621331 | 5.012401819 | 9.456386937 | 6.200909467 | 0.413393964 | 261.8850765 | 235.0144688 | 0.206696982 | 2.428689541 |
| ad- | | | | | | | | | | |
| justed | | | | | | | | | | |
| data | | | | | | | | | | |

Probe

| | 50 | 103 | 104 | 105 |
|---|---|---|---|---|
| Hyb | 0.051674246 | 8.009508061 | 21.65150889 | 0.568416701 |
| ad- | | | | |
| justed | | | | |
| data | | | | |

TABLE 7
Data set following normalisation

| | Probe | | | | | | | | | |
|---|---|---|---|---|---|---|---|---|---|---|
| | 0 | 1 | 2 | 3 | 4 | 5 | 6 | 7 | 8 | 9 |
| Nor- | 39.90146827 | 2.049095604 | 226.4348805 | 110.1761624 | 1.053325259 | 0.434377731 | 5.013782072 | 1.776521563 | 4.335974759 | 55.75664619 |
| mal- | | | | | | | | | | |
| ised | | | | | | | | | | |
| data | | | | | | | | | | |
| | Probe | | | | | | | | | |
| | 10 | 11 | 12 | 13 | 14 | 15 | 16 | 17 | 18 | 19 |
| Nor- | 46.20320406 | 1.067156835 | 2.956232674 | 14.28966269 | 164.7199777 | 1.69062125 | 1.752930721 | 12.71695229 | 1.494773273 | 38.19827022 |
| mal- | | | | | | | | | | |
| ised | | | | | | | | | | |
| data | | | | | | | | | | |
| | Probe | | | | | | | | | |
| | 20 | 21 | 22 | 23 | 24 | 25 | 26 | 27 | 28 | 29 |
| Nor- | 45.59413493 | 0.302402828 | 2.96922674 | 3.293599131 | 2.274834376 | 0.996537623 | 0.346558113 | 1.066689323 | 1.431661161 | 2.368829251 |
| mal- | | | | | | | | | | |
| ised | | | | | | | | | | |
| data | | | | | | | | | | |
| | Probe | | | | | | | | | |
| | 30 | 31 | 32 | 33 | 34 | 35 | 36 | 37 | 38 | 39 |
| Nor- | 97.0767168 | 32.21356304 | 1.70558488 | 0.086343829 | 0.186862151 | 19.53707819 | 1.531364753 | 6.975498538 | 0.103842051 | 26.25166552 |
| mal- | | | | | | | | | | |
| ised | | | | | | | | | | |
| data | | | | | | | | | | |
| | Probe | | | | | | | | | |
| | 40 | 41 | 42 | 43 | 44 | 45 | 46 | 47 | 48 | 49 |
| Nor- | 33.57748097 | 2.117601983 | 2.816319044 | 7.686220382 | 4.397992441 | 0.40089312 | 214.4184064 | 230.71127 | 0.131890189 | 2.232690034 |
| mal- | | | | | | | | | | |
| ised | | | | | | | | | | |
| data | | | | | | | | | | |

Probe

| | 50 | 103 | 104 | 105 |
|---|---|---|---|---|
| Nor- | 0.038252614 | 7.013373509 | 27.69737264 | 0.60197028 |
| mal- | | | | |
| ised | | | | |
| data | | | | |

TABLE 8
Data set checked for background

| | Probe | | | | | | | | | |
|---|---|---|---|---|---|---|---|---|---|---|
| | 0 | 1 | 2 | 3 | 4 | 5 | 6 | 7 | 8 | 9 |
| BG checked data | 39.90146827 | 0 | 226.4348805 | 110.1761624 | 0 | 0 | 0 | 0 | 0 | 55.75664619 |
| | Probe | | | | | | | | | |
| | 10 | 11 | 12 | 13 | 14 | 15 | 16 | 17 | 18 | 19 |
| BG checked data | 46.20320406 | 0 | 0 | 0 | 164.7199777 | 0 | 0 | 0 | 0 | 38.19827022 |
| | Probe | | | | | | | | | |
| | 20 | 21 | 22 | 23 | 24 | 25 | 26 | 27 | 28 | 29 |
| BG checked data | 45.59413493 | 0 | 0 | 0 | 0 | 0 | 0 | 0 | 0 | 0 |
| | Probe | | | | | | | | | |
| | 30 | 31 | 32 | 33 | 34 | 35 | 36 | 37 | 38 | 39 |
| BG checked data | 97.0767168 | 32.21356304 | 0 | 0 | 0 | 19.53707819 | 0 | 0 | 0 | 26.25166552 |
| | Probe | | | | | | | | | |
| | 40 | 41 | 42 | 43 | 44 | 45 | 46 | 47 | 48 | 49 |
| BG checked data | 33.57748097 | 0 | 0 | 0 | 0 | 0 | 214.4184064 | 230.71127 | 0 | 0 |

Probe

| | 50 | 103 | 104 | 105 |
|---|---|---|---|---|
| BG checked data | 0 | 0 | 27.69737264 | 0 |

TABLE 9
Data set following centering

| | Probe | | | | |
|---|---|---|---|---|---|
| | 0 | 1 | 2 | 3 | 4 |
| Centred | −27.02354384 | −12.62947631 | −28.507661 | −44.08041189 | −40.59134184 |
| data | | | | | |
| | Probe | | | | |
| | 5 | 6 | 7 | 8 | 9 |
| Centred | −12.27079637 | −21.76437749 | −46.35334588 | −73.46749748 | −51.51981803 |
| data | | | | | |
| | Probe | | | | |
| | 10 | 11 | 12 | 13 | 14 |
| Centred | −301.3688994 | −17.21722068 | −8.046376162 | −56.99541858 | −39.70909832 |
| data | | | | | |
| | Probe | | | | |
| | 15 | 16 | 17 | 18 | 19 |
| Centred | −44.67724211 | −0.926414605 | −38.19314123 | −154.2626382 | −22.98898606 |
| data | | | | | |
| | Probe | | | | |
| | 20 | 21 | 22 | 23 | 24 |
| Centred | −43.06202196 | −119.7774288 | −71.12408019 | −40.62803664 | −569.8209942 |
| data | | | | | |
| | Probe | | | | |
| | 25 | 26 | 27 | 28 | 29 |
| Centred | −1.989581656 | −1.713457687 | −87.14426606 | −0.100282038 | −67.94087802 |
| data | | | | | |
| | Probe | | | | |
| | 30 | 31 | 32 | 33 | 34 |
| Centred | −49.52345234 | −83.87950772 | −2.08008984 | 0 | −1.989008551 |
| data | | | | | |
| | Probe | | | | |
| | 35 | 36 | 37 | 38 | 39 |
| Centred | −27.44811268 | −39.2686822 | −171.9883911 | −37.67136638 | −11.79766251 |
| data | | | | | |
| | Probe | | | | |
| | 40 | 41 | 42 | 43 | 44 |
| Centred | −142.341303 | −24.07898983 | −645.760442 | −152.3347191 | −328.003766 |
| data | | | | | |
| | Probe | | | | |
| | 45 | 46 | 47 | 48 | 49 |
| Centred | −40.9444343 | −177.9405193 | −327.5029234 | −195.1030625 | −2.329866583 |
| data | | | | | |

Probe

| | 50 | 103 | 104 | 105 |
|---|---|---|---|---|
| Centred | −36.92478523 | −40.85323644 | −31.56030435 | −8.717386493 |
| data | | | | |

TABLE 10
Data set projected by 15 loading vector (Step (ii))

| | Loading vector | | | | | | | |
|---|---|---|---|---|---|---|---|---|
| | PC1 | PC2 | PC3 | PC4 | PC5 | PC6 | PC7 | PC8 |
| Data projected by | 619.2184977 | −402.8737744 | −207.5503637 | 91.30908749 | 183.3507408 | −77.14217491 | 543.9111741 | −142.5242892 |
| loading vector | | | | | | | | |

| | Loading vector | | | | | | |
|---|---|---|---|---|---|---|---|
| | PC9 | PC10 | PC11 | PC12 | PC13 | PC14 | PC15 |
| Data projected | 489.361778 | 172.0068772 | −137.6666566 | 0.432754519 | −113.8208536 | −104.5873989 | −40.45465493 |
| by loading vector | | | | | | | |

TABLE 11
Reinflated data set (Step (iii))

| | Probe | | | | |
|---|---|---|---|---|---|
| | 0 | 1 | 2 | 3 | 4 |
| Reinflated data | −16.28527981 | 4.282976317 | −21.0697846 | −11.87483876 | −30.26649115 |
| | Probe | | | | |
| | 5 | 6 | 7 | 8 | 9 |
| Reinflated data | 5.141867717 | −2.597888749 | −41.2071567 | −105.8671823 | −110.2352987 |
| | Probe | | | | |
| | 10 | 11 | 12 | 13 | 14 |
| Reinflated data | −271.7114099 | −47.26707379 | −13.36384232 | −3.708870897 | −23.22096946 |
| | Probe | | | | |
| | 15 | 16 | 17 | 18 | 19 |
| Reinflated data | −35.53919007 | −5.15748711 | −4.006900436 | −167.1165208 | −22.55549691 |
| | Probe | | | | |
| | 20 | 21 | 22 | 23 | 24 |
| Reinflated data | −59.84469431 | −147.4498659 | −80.69592993 | −42.95781022 | −591.2771802 |
| | Probe | | | | |
| | 25 | 26 | 27 | 28 | 29 |
| Reinflated data | −16.57525981 | −6.161210071 | −60.45008896 | −1.600920004 | −79.04167189 |
| | Probe | | | | |
| | 30 | 31 | 32 | 33 | 34 |
| Reinflated data | −68.74911311 | −110.6602281 | −4.444441798 | 2.678325 | 1.082427477 |
| | Probe | | | | |
| | 35 | 36 | 37 | 38 | 39 |
| Reinflated data | 0.27543201 | −70.5725879 | −177.80101 | −60.90536079 | −7.821457405 |
| | Probe | | | | |
| | 40 | 41 | 42 | 43 | 44 |
| Reinflated data | −153.4686911 | −20.35932606 | −629.1116591 | −133.4222904 | −314.7722453 |
| | Probe | | | | |
| | 45 | 46 | 47 | 48 | 49 |
| Reinflated data | −42.34584901 | −102.0087072 | −321.0313364 | −163.2957603 | −3.59112869 |

Probe

| | 50 | 103 | 104 | 105 |
|---|---|---|---|---|
| Reinflated data | −39.71174924 | −35.69931905 | −12.62559789 | −23.73199124 |

TABLE 12
Square of difference between centered data (Table 9) and reinflated data set (Table 11) (Step (iv))

| | Probe | | | | |
|---|---|---|---|---|---|
| | 0 | 1 | 2 | 3 | 4 |
| Difference | 10.73826403 | 16.91245262 | 7.437876402 | 32.20557313 | 10.32485069 |
| Square | 115.3103144 | 286.0310537 | 55.32200537 | 1037.19894 | 106.6025418 |
| | Probe | | | | |
| | 5 | 6 | 7 | 8 | 9 |
| Difference | 17.41266409 | 19.16648874 | 5.146189184 | −32.39968487 | −58.71548071 |
| Square | 303.2008705 | 367.3542906 | 26.48326312 | 1049.73958 | 3447.507675 |
| | Probe | | | | |
| | 10 | 11 | 12 | 13 | 14 |
| Difference | 29.65748947 | −30.04985311 | −5.31746616 | 53.28654769 | 16.48812886 |
| Square | 879.5666819 | 902.9936719 | 28.27544636 | 2839.456164 | 271.8583934 |
| | Probe | | | | |
| | 15 | 16 | 17 | 18 | 19 |
| Difference | 9.138052039 | −4.231072504 | 34.18624079 | −12.85388267 | 0.433489153 |
| Square | 83.50399506 | 17.90197454 | 1168.699059 | 165.2222997 | 0.187912846 |
| | Probe | | | | |
| | 20 | 21 | 22 | 23 | 24 |
| Difference | −16.78267235 | −27.67243716 | −9.57184974 | −2.329773577 | −21.45618596 |
| Square | 281.6580911 | 765.7637781 | 91.62030744 | 5.427844921 | 460.3679159 |
| | Probe | | | | |
| | 25 | 26 | 27 | 28 | 29 |
| Difference | −14.58567815 | −4.447752384 | 26.69417709 | −1.500637966 | −11.10079386 |
| Square | 212.7420072 | 19.78250127 | 712.5790907 | 2.251914306 | 123.2276244 |
| | Probe | | | | |
| | 30 | 31 | 32 | 33 | 34 |
| Difference | −19.22566077 | −26.78072039 | −2.364351958 | 2.67833E−22 | 3.071436028 |
| Square | 369.6260321 | 717.2069848 | 5.590160183 | 7.17343E−44 | 9.433719272 |
| | Probe | | | | |
| | 35 | 36 | 37 | 38 | 39 |
| Difference | 27.72354469 | −31.30390571 | −5.812618944 | −23.23399441 | 3.976205105 |
| Square | 768.5949302 | 979.9345125 | 33.78653898 | 539.8184964 | 15.81020704 |
| | Probe | | | | |
| | 40 | 41 | 42 | 43 | 44 |
| Difference | −11.12738806 | 3.719663769 | 16.64878298 | 18.9124287 | 13.23152074 |
| Square | 123.818765 | 13.83589856 | 277.1819746 | 357.6799594 | 175.0731412 |
| | Probe | | | | |
| | 45 | 46 | 47 | 48 | 49 |
| Difference | −1.401414709 | 75.93181214 | 6.471586973 | 31.80730217 | −1.261262107 |
| Square | 1.963963185 | 5765.640095 | 41.88143795 | 1011.704471 | 1.590782101 |

Probe

| | 50 | 103 | 104 | 105 |
|---|---|---|---|---|
| Difference | −2.786964008 | 5.153917393 | 18.93470646 | −15.01460475 |
| Square | 7.767168384 | 26.56286449 | 358.5231088 | 225.4383557 |

Sum of squared differences (Qres): 27656.3

Sum of values after eigenvalues applied to projected data set (Table 10): 77.30951 (Hotelling's T²; step (v))

Qres and Hotelling's T²values were then compared to predetermined normobiotic to dysbiotic threshold values: [T²=32.49 and Qres=42834.81]. Dysbiosis was confirmed as likely as T² value exceeds threshold.

T² and Qres were then combined into a single metric using squares; weights (0.938 and 0.157) and square root (i.e. Formula Ill). The resulting figure was 2318146

The resulting single metric was then plotted on numerical scale with a normobiotic to dysbiotic class separation point of 0.395 (representative value 2), and further thresholds at 1.632 (representative value 3), 2.492 (representative value 4) and infinity at a representative value of 5. This placed the sample between thresholds represented by the values 3 and 4 (close to the upper limit of 2.492 on the interval from 3 to 4). The precise location of the sample of this scale was then calculated as follows:
Total log normal distribution density area between 3 and 4 was calculated:
   0.6820813
   0.4840499
   0.1980315
   (first minus second equals third)
The log normal distribution density area between 3 and the sample was then calculated:
   0.6502034
   0.4840499
   0.1661535
   (first minus second equals third)
The log normal distribution density area between 3 and the sample was then divided by the log normal distribution density area between 3 and 4 to find the precise fraction: 0.8390257. The lower integer (3) was then added to get the precise position on the scale: 3.839026. This was then rounded up to 4 for reporting.

1. A method for determining the likelihood of GI tract dysbiosis in a subject, said method comprising:
(i) providing a test data set, wherein said test data set comprises at least one microbiota profile, said microbiota profile being a profile of the relative levels of a plurality of microorganisms or groups of microorganisms in a sample from the GI tract of the subject and wherein each level of each microorganism or group of microorganisms is a profile element of said test data set,
(ii) applying to said test data set at least one loading vector determined from latent variables within the profiles of the levels of said plurality of microorganisms or groups of microorganisms in corresponding GI tract samples from a plurality of normal subjects, thereby producing a first projected data set,
(iii) applying to said first projected data set a transposed version of said at least one loading vector, thereby producing a second projected data set,
(iv) comparing said test data set with said second projected data set and combining the differences between the corresponding profile elements of the second projected data set and the test data set and comparing the combined differences with a normobiotic to dysbiotic threshold value determined from the corresponding analysis of said plurality of microorganisms or groups of microorganisms in corresponding GI tract samples from a plurality of normal subjects and/or subjects with dysbiosis,
(v) applying at least one eigenvalue to said first projected data set, said eigenvalue determined from said at least one loading vector, and combining the resulting values for each profile element and comparing the combined values with a normobiotic to dysbiotic threshold value determined from the corresponding analysis of said plurality of microorganisms or groups of microorganisms in corresponding GI tract samples from a plurality of normal subjects and/or subjects with dysbiosis,
wherein step (v) may be performed before or after or concurrently with either of steps (iii) or (iv), and wherein a microbiota profile with said combined differences or said combined resulting values in excess of said respective normobiotic to dysbiotic thresholds is indicative of a likelihood of dysbiosis.

2. The method of claim 1, wherein the combination of each difference between corresponding elements in step (iv) comprises calculating the square of each said difference and then the squared values are summed.

3. The method of claim 1, wherein the combination of each resulting value in step (v) comprises calculating the square of each resulting value and then the squared values are summed.

4. The method of claim 1, wherein said method comprises:
providing a test data set, wherein said test data set comprises at least one microbiota profile, said microbiota profile being a profile of the relative levels of a plurality of microorganisms or groups of microorganisms in a sample from the GI tract of the subject and wherein each level of each microorganism or group of microorganisms is a profile element of said test data set,
(ii) applying to said test data set at least one loading vector determined from latent variables within the profiles of the levels of said plurality of microorganisms or groups of microorganisms in corresponding GI tract samples from a plurality of normal subjects, thereby producing a first projected data set,
(iii) providing said first projected data set,
(iv) from said first projected data set calculating the Q-residual of the microbiota profile and comparing the Q-residual of the microbiota profile with a normobiotic to dysbiotic threshold Q-residual value determined from the corresponding analysis of said plurality of microorganisms or groups of microorganisms in corresponding GI tract samples from a plurality of normal subjects and/or subjects with dysbiosis,
(v) from said first projected data set calculating the Hotelling's T² for the microbiota profile from the variance explained by the latent variables of step (ii) and comparing said Hotelling's T² for the microbiota profile with a normobiotic to dysbiotic threshold Hotelling's T² value determined from the corresponding analysis of said plurality of microorganisms or groups of microorganisms in corresponding GI tract samples from a plurality of normal subjects and/or a plurality of subjects with dysbiosis,
wherein step (v) may be performed before or after or concurrently with step (iv), and wherein a microbiota profile with a Q-residual or Hotelling's T² in excess of said respective thresholds is indicative of a likelihood of dysbiosis.

5. The method of claim 1, wherein said method further comprises a preceding step in which at least one of said microbiota profiles is prepared.

6. The method of claim 1, wherein said test data set comprises a plurality of microbiota profiles and said test data set is arranged into a matrix.

7. The method of claim 1, wherein the latent variables comprise at least one orthogonal latent variable, preferably are all orthogonal latent variables.

8. The method of claim 7, wherein said orthogonal latent variables are determined by the orthogonal transformation into principle components of the levels of said plurality of microorganisms or groups of microorganisms in GI tract samples from a plurality of normal subjects.

9. The method of claim 8, wherein the orthogonal transformation into principle components is by at least one of partial least squares regression analysis, Principle Component Analysis, canonical correlation analysis, redundancy analysis, correspondence analysis, and canonical correspondence analysis.

10. The method of claim 1, wherein at least 2 loading vectors, preferably at least 3, 5, 7, 9, 11, 13, 15, 17, 19 or 20 loading vectors, and/or no more than 50 loading vectors, preferably no more than 40, 30, 25, 20, or 15 loading vectors are applied.

11. The method of claim 1, wherein the loading vector is applied in the form of a projection matrix.

12. The method of claim 1, wherein said microbiota profiles are quantitative or semi-quantitative and wherein said method provides a quantitative or semi-quantitative measure of the extent of dysbiosis.

13. A method for quantifying dysbiosis, said method comprising performing the method of claim 12, wherein said comparisons with normobiotic to dysbiotic thresholds together comprise combining the combination of differences between corresponding profile elements in step (iv) and the combination of resulting values in step (v) into a single metric for dysbiosis.

14. The method of claim 13, wherein the Euclidean distance from the origin for both the combination of differences between corresponding profile elements in step (iv) and the combination of resulting values in step (v) is calculated.

15. The method of claim 14, wherein the combination of differences between corresponding profile elements in step (iv) is expressed as Q-residuals and the combination of resulting values in step (v) is expressed as Hotelling's T² and wherein the Euclidean distance from the origin for both Q-residuals and Hotelling's T² is calculated with Formula I:
r=\sqrt{{T²}²+Qres²}

16. The method of claim 13, wherein the combining of the combination of differences between corresponding profile elements in step (iv) and the combination of resulting values in step (v) into a single metric for dysbiosis comprises scaling said combination of differences between corresponding profile elements in step (iv) and the combination of resulting values in step (v) to result in values of similar magnitude.

17. The method of claim 13, wherein said single metric is plotted on a finite numerical scale with a normobiosis to dysbiosis class separation at a predetermined point on said finite numerical scale which represents, or is, a combination of the normobiotic to dysbiotic class thresholds of steps (iv) and (v), similarly scaled if scaling has been applied.

18. The method of claim 13, wherein said single metric is plotted on a finite numerical scale with a normobiosis to dysbiosis class separation at a predetermined point on said finite numerical scale, and wherein
(a) for a test sample having at least one of the combination of differences between corresponding profile elements in step (iv) or the combination of resulting values in step (v) above the normobiotic to dysbiotic class threshold values of steps (iv) and (v), respectively, said class separation point corresponds to that of one or other of the exceeded normobiotic to dysbiotic class threshold value of steps (iv) or (v), similarly scaled if scaling has been applied, and
(b) for a test sample in which neither of the combination of differences between corresponding profile elements in step (iv) or the combination of resulting values in step (v) are beyond the normobiotic to dysbiotic threshold values of steps (iv) and (v), respectively, said class separation point corresponds to the sum of the normobiotic to dysbiotic class thresholds of steps (iv) and (v), similarly scaled if scaling has been applied.

19. The method of claim 13, wherein weightings are applied to the combination of differences between corresponding profile elements in step (iv) and the combination of resulting values in step (v) during the second combination step, and wherein said weightings minimise the effects of technical variation.

20. A method for obtaining information relevant to the diagnosis, monitoring and/or characterisation of diseases and conditions associated with perturbations in the microbiota of the GI tract or the assessment of the risk of developing a disease or condition which is associated with a perturbation of the microbiota profile of the GI tract, said method comprising performing a method as defined in claim 1, wherein the results of said method as defined above provides said information.

21. A method for diagnosing, monitoring and/or characterising diseases and conditions associated with perturbations in the microbiota of the GI tract or the assessing of the risk of developing a disease or condition which is associated with a perturbation of the microbiota profile of the GI tract, said method comprising performing a method as defined in any one of claim 1, wherein the indication the likelihood of dysbiosis or the extent of dysbiosis is indicative of the presence or absence, the risk of developing, the progress of, or the characteristics of said disease or condition associated with perturbations in the microbiota of the GI tract.

22. The method of claim 20, wherein said disease or condition associated with a perturbation in the microbiota of the GI tract is selected from functional GI tract disorders, small bowel bacterial overgrowth syndrome, GI tract cancers, breast cancer, ankylosing spondylitis; non-alcoholic steatohepatitis; atopic diseases, metabolic disorders, neurological disorders, autoimmune diseases, malnutrition, chronic fatigue syndrome and autism

23. The method of claim 22, wherein the functional GI tract disorder is IBS.

24. The method of claim 5, wherein said step of preparing said microbioata profiles comprises nucleic acid analysis, preferably nucleic acid sequencing, oligonucleotide probe hybridisation, primer based nucleic acid amplification;
antibody or other specific affinity ligand based detection; proteomic analysis or metabolomic analysis.

25. The method of claim 1, wherein the sample from the GI tract is selected from
(a) luminal contents of the GI tract, preferably stomach contents, intestinal contents, mucus and faeces/stool, or combinations thereof,
(b) parts of the mucosa, the submucosa, the muscularis externa, the adventitia and/or the serosa of a GI tract tissue/organ,
(c) nucleic acid prepared from (a) or (b), preferably by reverse transcription and/or nucleic acid amplification, or
(d) a microbial culture of (a) or (b).

26. The method of claim 25, wherein said GI tract sample is obtained from the jejunum, the ileum, the cecum, the colon, the rectum or the anus.

27. A computer, system or apparatus carrying a program adapted to perform the method of claim 1.

28. The system or apparatus of claim 27, further adapted to perform microbiota profiling or a step thereof.
